(19) United States
(12) Patent Application Publication
(10) Pub. No.: US 2026/0233991 A1
(43) Pub. Date: Aug. 13, 2026

(54) HYDROGEN CARRIER MANUFACTURING SYSTEM, CONTROL APPARATUS, AND HYDROGEN CARRIER MANUFACTURING METHOD

(71) Applicant: ENEOS Corporation, Tokyo (JP)

(72) Inventors: Jun INOUE, Tokyo (JP); Seiji MAEDA, Tokyo (JP); Yoshinori NEGISHI, Tokyo (JP); Kenji MACHII, Tokyo (JP); Tadashi SEIKE, Tokyo (JP); Ai MINODA, Tokyo (JP)

(21) Appl. No.: 19/156,626

(22) PCT Filed: Feb. 19, 2024
(86) PCT No.: PCT/JP2024/005686

§ 371 Date: Aug. 14, 2025

(30) Foreign Application Priority Data
Feb. 21, 2023 (JP) 2023-025202

(51) Int. Cl. C01B 3/0018; C01B 3/0015

(52) U.S. Cl. CPC C01B 3/0018; C01B 3/0015; C01B 2203/1676; C01B 2203/1685

(57) ABSTRACT

A hydrogen carrier manufacturing system includes a hydrogen manufacturing device configured to manufacture hydrogen by using power; a hydrogen tank configured to store the hydrogen manufactured by the hydrogen manufacturing device; and a plurality of hydrogen carrier manufacturing devices configured to convert the hydrogen stored in the hydrogen tank into different types of hydrogen carriers.

### TECHNICAL FIELD

The present invention relates to a hydrogen carrier manufacturing system, a control apparatus, and a hydrogen carrier manufacturing method.

### BACKGROUND ART

In recent years, environmental problems such as global warming have become a global issue, and hydrogen, which does not generate carbon dioxide when used, is attracting attention as a new energy to promote decarbonization. In particular, if hydrogen is manufactured by using renewable energy such as solar power generation and wind power generation, further reduction of carbon dioxide can be expected.

Technology for efficiently storing and transporting hydrogen is required. Substances capable of storing and transporting hydrogen are called hydrogen carriers, etc. Various technologies for converting gaseous hydrogen (hydrogen gas) into hydrogen carriers have been proposed.

For example, Patent Document 1 discloses a solar power generation system having a hydrogen manufacturing means for storing solar energy. The solar power generation system disclosed in Patent Document 1 includes a hydrogen manufacturing means for manufacturing hydrogen by using a DC current obtained by a solar power generation device, and a hydrogen storage means for converting the hydrogen obtained by the hydrogen manufacturing means into saturated hydrocarbons and storing them.

### CITATION LIST

### Patent Documents

Patent Document 1: Japanese Unexamined Patent Application Publication No. 2014-203274

### SUMMARY OF INVENTION

### Technical Problem

However, there is a problem that the conventional technology does not consider manufacturing a plurality of types of hydrogen carriers in parallel. If a plurality of types of hydrogen carriers are manufactured in parallel, the operating rate of the entire manufacturing facility can be improved while applying the economic efficiency of each type of hydrogen carrier.

In view of the above technical problem, an object of an aspect of the present invention is to manufacture a plurality of types of hydrogen carriers in parallel.

### Solution to Problem

A hydrogen carrier manufacturing system according to an aspect of the present invention includes a hydrogen manufacturing device configured to manufacture hydrogen by using power; a hydrogen tank configured to store the hydrogen manufactured by the hydrogen manufacturing device; and a plurality of hydrogen carrier manufacturing devices configured to convert the hydrogen stored in the hydrogen tank into different types of hydrogen carriers.

### BRIEF DESCRIPTION OF THE DRAWINGS

FIG. 1 is a block diagram illustrating an example of a hydrogen supply chain.

FIG. 2 is a block diagram illustrating an example of the overall configuration of a hydrogen carrier manufacturing system.

FIG. 3 is a block diagram illustrating an example of a hardware configuration of a computer.

FIG. 4 is a block diagram illustrating an example of the functional configuration of a control apparatus.

FIG. 5 is a flowchart illustrating an example of a setting method.

FIG. 6 is a graph illustrating an example of a power market price profile.

FIG. 7 is a flowchart illustrating an example of a control method.

FIG. 8 is a flowchart illustrating an example of operation state determination processing.

FIG. 9 is a flowchart illustrating an example of operation state determination processing.

FIG. 10 is a flowchart illustrating an example of operation state determination processing.

FIG. 11 is a flowchart illustrating an example of operation state determination processing.

FIG. 12 is a flowchart illustrating an example of operation state determination processing.

FIG. 13 is a block diagram illustrating an example of the overall configuration of the hydrogen gas generation system in the modified example.

FIG. 14 is a block diagram illustrating an example of the functional configuration of the control apparatus in the modified example 1.

FIG. 15 is a block diagram illustrating an example of the functional configuration of the control apparatus in the modified example 2.

### DESCRIPTION OF EMBODIMENTS

Each embodiment of the present invention will be described below with reference to the attached drawings. In the present specification and the drawings, components having substantially the same functional configuration are denoted by the same reference numerals, and duplicate descriptions are omitted.

### Embodiment

The embodiment of the present invention is a hydrogen carrier manufacturing system for manufacturing hydrogen carriers. The hydrogen carriers are substances obtained by converting gaseous hydrogen (hydrogen gas) into a liquid or the like that can be efficiently stored and transported. In the hydrogen carrier manufacturing system of the present embodiment, hydrogen gas is manufactured by electrolyzing water, and the hydrogen gas is converted into a liquid to manufacture the hydrogen carriers. Hereinafter, simply referring to “hydrogen” means gaseous hydrogen (hydrogen gas).

### Hydrogen Carriers

Substances that can be used as hydrogen carriers are, for example, methylcyclohexane (MCH), ammonia (NH3), or liquid hydrogen (LH2). Methylcyclohexane can be obtained by reacting toluene with hydrogen. Ammonia can be obtained by reacting hydrogen with nitrogen. Liquid hydrogen is liquefied by cooling hydrogen to below the boiling point (−253° C.).

The hydrogen carriers can be converted back to hydrogen by a chemical reaction such as dehydrogenation, or the hydrogen carriers can be used as is (in the state of hydrogen carriers). Toluene obtained by dehydrogenation of methylcyclohexane can be transported to the manufacturing area of hydrogen carriers and reused for the manufacturing of new methylcyclohexane. Ammonia can be used as a raw material for fertilizers and chemical products, for example. Ammonia itself can also be used as a fuel. Liquid hydrogen can be used as a fuel for rockets, for example.

In the hydrogen carrier manufacturing system of the present embodiment, hydrogen is manufactured by using power derived from renewable energy. In the hydrogen carrier manufacturing system of the present embodiment, hydrogen is converted into hydrogen carriers by using power derived from renewable energy. Therefore, the hydrogen carriers manufactured by the hydrogen carrier manufacturing system of the present embodiment can be used as clean energy with reduced emission of carbon dioxide in the cycle from manufacturing to use.

### Hydrogen Supply Chain

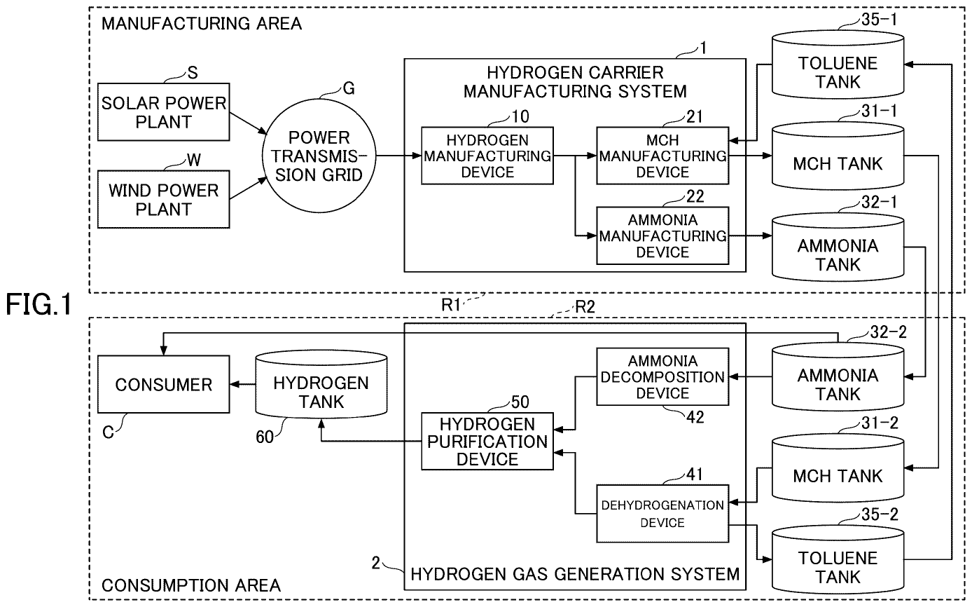

The hydrogen supply chain of the present embodiment will be described with reference to FIG. 1. The hydrogen supply chain is a series of flows from manufacturing, transportation, and consumption of hydrogen.

The hydrogen supply chain of the present embodiment will be described with reference to FIG. 1. FIG. 1 illustrates an example of a hydrogen supply chain in which methylcyclohexane and ammonia are manufactured in parallel as hydrogen carriers.

As illustrated in FIG. 1, the hydrogen supply chain in the present embodiment includes a manufacturing area R1 and a consumption area R2. At least one of hydrogen or hydrogen carriers (hereinafter abbreviated as “hydrogen and the like”) is manufactured at the manufacturing area R1. Hydrogen and the like are consumed at the consumption area R2. The manufacturing area R1 and the consumption area R2 may be different countries or different regions within the same country. At least one of the manufacturing area RI or the consumption area R2 may be plural. The transport route of the hydrogen carriers connecting the manufacturing area R1 and the consumption area R2 may be a land route only, or may include a sea route or an air route.

A hydrogen carrier manufacturing system 1, an MCH tank 31-1, an ammonia tank 32-1, and a toluene tank 35-1 are installed in the manufacturing area R1. Methylcyclohexane (an example of the first hydrogen carrier) manufactured by the hydrogen carrier manufacturing system 1 is stored in the MCH tank 31-1. Ammonia (an example of the second hydrogen carrier) manufactured by the hydrogen carrier manufacturing system 1 is stored in the ammonia tank 32-1. Toluene used to manufacture methylcyclohexane is stored in the toluene tank 35-1.

The hydrogen carrier manufacturing system 1 is connected to the power transmission grid G. A solar power plant S and a wind power plant W supplying power derived from renewable energy are connected to the power transmission grid G. A thermal power plant or a nuclear power plant supplying power derived from energy other than renewable energy may be connected to the power transmission grid G. The hydrogen carrier manufacturing system 1 receives power derived from renewable energy from the solar power plant S or the wind power plant W via the power transmission grid G.

The hydrogen carrier manufacturing system 1 manufactures methylcyclohexane and ammonia by using power supplied from the power transmission grid G. The hydrogen carrier manufacturing system 1 includes a hydrogen manufacturing device 10, a MCH manufacturing device 21 (an example of a first hydrogen carrier manufacturing device), and an ammonia manufacturing device 22 (an example of a second hydrogen carrier manufacturing device). A hydrogen manufacturing device 10 manufactures hydrogen by using power supplied from a power transmission grid G.

The MCH manufacturing device 21 reacts hydrogen manufactured by the hydrogen manufacturing device 10 with toluene supplied from a toluene tank 35-1 to manufacture methylcyclohexane. Methylcyclohexane manufactured by the MCH manufacturing device 21 is stored in a MCH tank 31-1.

The ammonia manufacturing device 22 reacts nitrogen in the atmosphere with hydrogen manufactured by the hydrogen manufacturing device 10 to manufacture ammonia. The nitrogen can be obtained by an air separating device or the like. Ammonia manufactured by the ammonia manufacturing device 22 is stored in an ammonia tank 32-1.

Methylcyclohexane stored in the MCH tank 31-1 and ammonia stored in the ammonia tank 32-1 are transported from the manufacturing area R1 to the consumption area R2 by means of transport corresponding to the transport route between the manufacturing area R1 and the consumption area R2. For transportation of methylcyclohexane and ammonia, for example, a tanker or the like is used for the sea route, and a tank truck or the like is used by land route. However, transportation means are not limited to these, and any means that can safely transport methylcyclohexane and ammonia may be used.

The hydrogen gas generation system 2, the MCH tank 31-2, the ammonia tank 32-2, the toluene tank 35-2, and the hydrogen tank 60 are installed in the consumption area R2. Methylcyclohexane transported from the manufacturing area R1 is stored in the MCH tank 31-2. Ammonia transported from the manufacturing area R1 is stored in the ammonia tank 32-2. Toluene obtained by dehydrogenating methylcyclohexane is stored in the toluene tank 35-2. Toluene stored in the toluene tank 35-2 is transported from the consumption area R2 to the manufacturing area R1 by means of transport corresponding to the transport route between the manufacturing area R1 and the consumption area R2. Toluene transported to the manufacturing area R1 is stored in the toluene tank 35-1.

The hydrogen gas generation system 2 converts methylcyclohexane supplied from the MCH tank 31-2 and ammonia supplied from the ammonia tank 32-2 back to hydrogen. The hydrogen gas generation system 2 includes a dehydrogenation device 41, an ammonia decomposition device 42, and a hydrogen purification device 50.

The dehydrogenation device 41 separates methylcyclohexane supplied from the MCH tank 31-2 into hydrogen and toluene by a chemical reaction such as dehydrogenation. Toluene obtained by the dehydrogenation device 41 is stored in a toluene tank 35-2.

The ammonia decomposition device 42 reacts ammonia supplied from the ammonia tank 32-2 with a catalyst and separates it into hydrogen and nitrogen. The ammonia decomposition device 42 may convert ammonia into ammonia gas by vaporizing ammonia.

The hydrogen purification device 50 purifies hydrogen obtained by the dehydrogenation device 41 or the ammonia decomposition device 42 into hydrogen of high purity. Hydrogen purified by the hydrogen purification device 50 is stored in a hydrogen tank 60.

Hydrogen stored in the hydrogen tank 60 is supplied to the consumer C and consumed. The supply to the consumer C may be carried by filling a container such as a hydrogen cylinder, or may be transported via a preinstalled pipeline.

Ammonia gas obtained by the ammonia decomposition device 42 is supplied to the consumer C and consumed. Ammonia stored in the ammonia tank 32-2 may be supplied to the consumer C as ammonia and consumed.

The consumer C may be, for example, a steel plant, a power plant, a chemical plant, or a hydrogen station. The consumer C may be the hydrogen gas generation system 2 itself. That is, hydrogen or ammonia obtained in the hydrogen gas generation system 2 may be consumed in the hydrogen gas generation system 2 or in other systems attached to the hydrogen gas generation system 2.

The hydrogen carrier manufacturing system 1 in the present embodiment may manufacture liquid hydrogen instead of methylcyclohexane or ammonia or in addition to methylcyclohexane and ammonia. When liquid hydrogen is manufactured as a hydrogen carrier, a liquid hydrogen tank is installed in the manufacturing area R1. The hydrogen carrier manufacturing system 1 is provided with a liquid hydrogen manufacturing device. The liquid hydrogen manufacturing device cools hydrogen manufactured by the hydrogen manufacturing device 10 to manufacture liquid hydrogen. The liquid hydrogen manufactured by the liquid hydrogen manufacturing device is stored in a liquid hydrogen tank.

When liquid hydrogen is manufactured as a hydrogen carrier, a liquid hydrogen tank is installed in the consumption area R2. The hydrogen gas generation system 2 is provided with a liquid hydrogen vaporizing device. The liquid hydrogen vaporizing device vaporizes liquid hydrogen supplied from the liquid hydrogen tank and converts it back to hydrogen. The hydrogen obtained by the liquid hydrogen vaporizing device is supplied to the user C and consumed. The liquid hydrogen stored in the liquid hydrogen tank may be supplied to the user C as liquid hydrogen and consumed. The hydrogen or liquid hydrogen may be consumed inside the hydrogen gas generation system 2 or in another system attached to the hydrogen gas generation system 2.

### Overall Configuration of the Hydrogen Carrier Manufacturing System

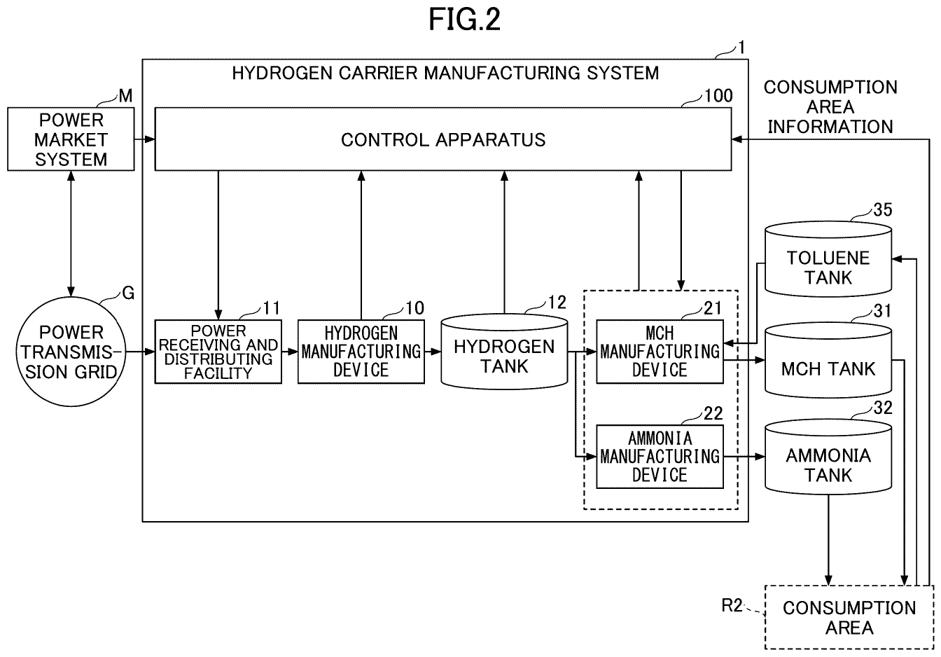

The overall configuration of the hydrogen carrier manufacturing system of the present embodiment will be described with reference to FIG. 2. FIG. 2 is a block diagram illustrating an example of the overall configuration of the hydrogen carrier manufacturing system of the present embodiment.

As illustrated in FIG. 2, the hydrogen carrier manufacturing system 1 of the present embodiment includes a hydrogen manufacturing device 10, a power receiving and distributing facility 11, a hydrogen tank 12, a MCH manufacturing device 21, an ammonia manufacturing device 22, and a control apparatus 100. The hydrogen carrier manufacturing system 1 in the present embodiment is connected to the power market system M, the power transmission grid G, the MCH tank 31, the ammonia tank 32, and the toluene tank 35 existing at the manufacturing area R1.

The power market system M is an information processing system that manages the power market. In the power market, the power supplied by the power transmission grid G is traded. The power market system M determines the price of power according to the demand and supply of power. In the present embodiment, the power market system M determines the price of power for each power generation system. For example, the power market system M determines different prices for power derived from renewable energy (for example, solar, wind, geothermal, or biomass) and power derived from energy other than renewable energy (for example, coal, oil, natural gas, or nuclear power).

The power receiving and distributing facility 11 receives power supplied from the power transmission grid G and distributes power to each device of the hydrogen carrier manufacturing system 1. The power receiving and distributing facility 11 can set the power to be distributed to each device. The power receiving and distributing facility 11 sets at least the power input to the hydrogen manufacturing device 10 based on the signal received from the control apparatus 100.

The hydrogen manufacturing device 10 manufactures hydrogen by using the power input from the power receiving and distributing facility 11. In the present embodiment, the hydrogen manufacturing device 10 manufactures hydrogen by electrolyzing water stored in the water electrolyzer. The hydrogen manufactured by the hydrogen manufacturing device 10 is stored in the hydrogen tank 12.

The amount of hydrogen manufactured by the hydrogen manufacturing device 10 varies according to the amount of input power. Therefore, the amount of hydrogen manufactured by the hydrogen manufacturing device 10 can be changed by changing the amount of power input from the power receiving and distributing facility 11 to the hydrogen manufacturing device 10.

The hydrogen manufactured by the hydrogen manufacturing device 10 is stored in the hydrogen tank 12. Hydrogen stored in the hydrogen tank 12 is supplied to a MCH manufacturing device 21 and an ammonia manufacturing device 22.

The MCH manufacturing device 21 reacts hydrogen supplied from the hydrogen tank 12 with toluene supplied from the toluene tank 35 and converts it into methylcyclohexane. The methylcyclohexane manufactured by the MCH manufacturing device 21 is stored in the MCH tank 31.

The ammonia manufacturing device 22 reacts hydrogen manufactured by the hydrogen manufacturing device 10 with nitrogen obtained by an air separating device or the like to manufacture ammonia. The ammonia manufactured by the ammonia manufacturing device 22 is stored in the ammonia tank 32.

The control apparatus 100 is an information processing apparatus such as a personal computer, workstation, or server that controls the operation of each device included in the hydrogen carrier manufacturing system 1. The control apparatus 100 is configured to be capable of data communication with the power market system M, the power receiving and distributing facility 11, the hydrogen manufacturing device 10, the hydrogen tank 12, the MCH manufacturing device 21, and the ammonia manufacturing device 22 through a communication network.

The control apparatus 100 controls the operation of each device included in the hydrogen carrier manufacturing system 1. The control apparatus 100 generates information (hereinafter also referred to as “control information”) for controlling each device on the basis of information about the consumption area R2 at which the hydrogen carrier is consumed (hereinafter also referred to as “consumption area information”). Based on the generated control information, the control apparatus 100 transmits control signals for controlling the operation of at least the power receiving and distributing facility 11, the MCH manufacturing device 21, and the ammonia manufacturing device 22.

The overall configuration of the hydrogen carrier manufacturing system 1 illustrated in FIG. 2 is an example, and various system configurations can be used depending on the application and purpose. For example, one or more of the hydrogen manufacturing device 10, the MCH manufacturing device 21, the ammonia manufacturing device 22, and the control apparatus 100 may be included in the hydrogen carrier manufacturing system 1 by being provided in plurality. For example, the control apparatus 100 may be implemented by a plurality of computers or as a cloud computing service.

The division of devices such as the hydrogen manufacturing device 10, the MCH manufacturing device 21, the ammonia manufacturing device 22, and the control apparatus 100 illustrated in FIG. 2 is an example.

### Hardware Configuration of the Hydrogen Carrier Manufacturing System

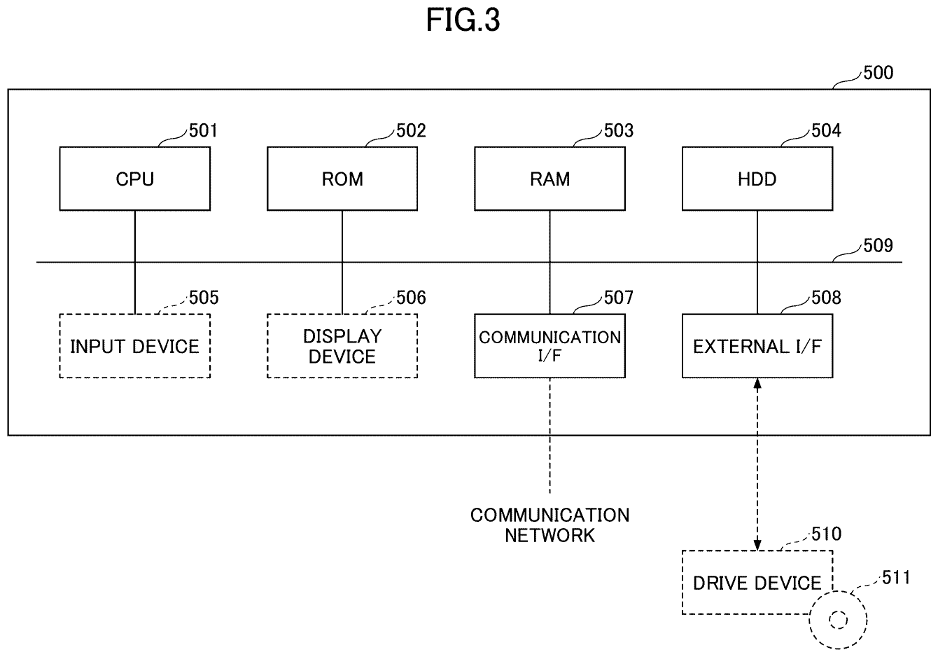

The hardware configuration of each device included in the hydrogen carrier manufacturing system 1 of the present embodiment will be described with reference to FIG. 3.

### Hardware Configuration of the Computer

The control apparatus 100 of the present embodiment is implemented by, for example, a computer. FIG. 3 is a block diagram illustrating an example of the hardware configuration of the computer in the present embodiment.

As illustrated in FIG. 3, a computer 500 in the present embodiment includes a CPU (Central Processing Unit) 501, a ROM (Read Only Memory) 502, a RAM (Random Access Memory) 503, an HDD (Hard Disk Drive) 504, an input device 505, a display device 506, a communication I/F (interface) 507, and an external I/F 508. The CPU 501, the ROM 502, and the RAM 503 form what is referred to as a computer. The pieces of hardware of the computer 500 are connected to each other via a bus line 509. The input device 505 and the display device 506 may be connected to the external I/F 508 for use.

The CPU 501 is an arithmetic unit that loads programs and data from a storage device such as the ROM 502 or the HDD 504 onto the RAM 503 and executes processing to implement the control and functions of the entire computer 500. The computer 500 may have a GPU (Graphics Processing Unit) in addition to the CPU 501 or instead of the CPU 501.

The ROM 502 is an example of a nonvolatile semiconductor memory (storage device) that can retain programs and data even when the power is turned off. The ROM 502 functions as a main storage device that stores various programs and data necessary for the CPU 501 to execute various programs installed in the HDD 504. Specifically, the ROM 502 stores boot programs such as BIOS (Basic Input/Output System) and EFI (Extensible Firmware Interface) that are executed when the computer 500 is started, and data such as OS (Operating System) settings and network settings.

The RAM 503 is an example of a volatile semiconductor memory (storage device) in which programs and data are erased when the power is turned off. The RAM 503 is, for example, a DRAM (Dynamic Random Access Memory) or a SRAM (Static Random Access Memory). The RAM 503 provides a work area that is loaded when various programs installed in the HDD 504 are executed by the CPU 501.

The HDD 504 is an example of a nonvolatile storage device that stores programs and data. The programs and data stored in the HDD 504 include the OS, which is the basic software that controls the entire computer 500, and applications that provide various functions on the OS. The computer 500 may use a storage device (e.g., SSD: Solid State Drive, etc.) using a flash memory as a storage medium instead of the HDD 504.

The input device 505 includes a touch panel, operation keys and buttons, a keyboard and a mouse used by the user to input various signals, and a microphone to input sound data such as sound.

The display device 506 includes a liquid crystal for displaying a screen, a display such as organic EL (Electro-Luminescence), and a speaker for outputting sound data such as sound.

The communication I/F 507 is an interface for connecting to a communication network for the computer 500 to perform data communication.

The external I/F 508 is an interface with an external device. The external device includes a drive device 510.

The drive device 510 is a device for setting the recording medium 511. The recording medium 511 here includes a medium for recording information optically, electrically, or magnetically, such as a CD-ROM, a flexible disk, a magneto-optical disk, or the like. The recording medium 511 may also include a semiconductor memory for recording information electrically, such as a ROM, a flash memory, or the like. Thus, the computer 500 can read and/or write data from/in the recording medium 511 via the external I/F 508.

Various programs installed on the HDD 504 are installed by, for example, setting the distributed recording medium 511 to the drive device 510 connected to the external I/F 508, and reading various programs recorded on the recording medium 511 by the drive device 510. Alternatively, various programs installed on the HDD 504 may be installed by being downloaded from another network different from the communication network via the communication I/F 507.

### Functional Configuration of the Hydrogen Carrier Manufacturing System

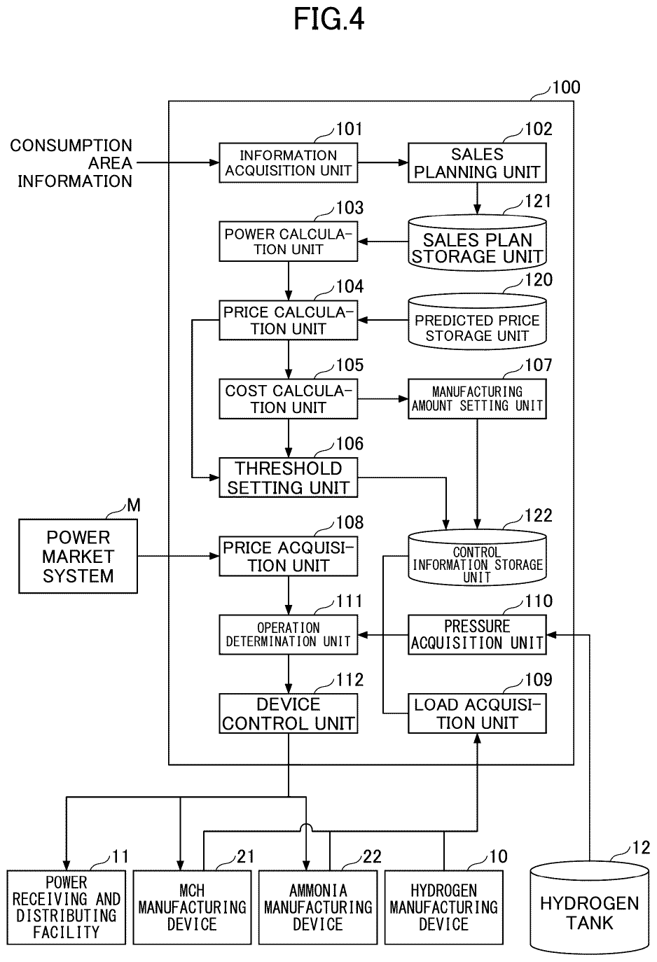

The functional configuration of the hydrogen carrier manufacturing system in the present embodiment will be described with reference to FIG. 4. FIG. 4 is a block diagram illustrating an example of the functional configuration of the hydrogen carrier manufacturing system 1 in the present embodiment.

### Functional Configuration of the Control Apparatus

As illustrated in FIG. 4, the control apparatus 100 in the present embodiment includes an information acquisition unit 101, a sales planning unit 102, a power calculation unit 103, a price calculation unit 104, a cost calculation unit 105, a threshold setting unit 106, a manufacturing amount setting unit 107, a price acquisition unit 108, a load acquisition unit 109, a pressure acquisition unit 110, an operation determination unit 111, an device control unit 112, a predicted price storage unit 120, a sales plan storage unit 121, and a control information storage unit 122.

The information acquisition unit 101, the sales planning unit 102, the power calculation unit 103, the price calculation unit 104, the cost calculation unit 105, the threshold setting unit 106, the manufacturing amount setting unit 107, the price acquisition unit 108, the load acquisition unit 109, the pressure acquisition unit 110, the operation determination unit 111, and the device control unit 112 are implemented by, for example, processing that the CPU 501 is caused to execute by a program loaded on the RAM 503 from the HDD 504 illustrated in FIG. 3.

The predicted price storage unit 120, the sales plan storage unit 121, and the control information storage unit 122 are implemented by using, for example, the HDD 504 illustrated in FIG. 3.

The predicted price storage unit 120 stores information (hereinafter also referred to as “power market price profile”) indicating the predicted value of the market price (hereinafter also referred to as “power market price”) at which power derived from renewable energy is traded in the power market. The power market price is determined at predetermined time intervals (for example, every five minutes) in the power market system M. The power market price profile is information predicting the frequency distribution of the power market price during a predetermined plan period (for example, 1 month).

The sales plan storage unit 121 stores information indicating the sales plan of the hydrogen carrier manufactured by the hydrogen carrier manufacturing system 1. The sales plan in the present embodiment includes the sales amount and sales price of the hydrogen carrier in a predetermined plan period. Information indicating the sales plan stored in the sales plan storage unit 121 is generated by the sales planning unit 102.

The control information storage unit 122 stores control information for controlling each device included in the hydrogen carrier manufacturing system 1. The control information stored in the control information storage unit 122 is generated by the threshold setting unit 106.

The control information in the present embodiment includes a threshold for determining whether to purchase the power for manufacturing the hydrogen carrier. The threshold is, for example, an upper limit price for the power market price of the power derived from renewable energy. Hereinafter, the upper limit price for the power market price is referred to as the “upper limit power price”.

The control information in the present embodiment further includes a stable operation range (hereinafter also referred to as “hydrogen manufacturing load range”) for the load (hereinafter also referred to as “hydrogen manufacturing load”) of the hydrogen manufacturing device 10. The hydrogen manufacturing load range is defined by the upper and lower limits of the hydrogen manufacturing load.

The control information in the present embodiment further includes a stable operation range (hereinafter also referred to as “first hydrogen carrier production load range”) for the load (hereinafter also referred to as “first hydrogen carrier production load”) of the MCH manufacturing device 21. The first hydrogen carrier production load range is defined by the upper and lower limits of the first hydrogen carrier production load.

The control information in the present embodiment further includes a stable operation range (hereinafter also referred to as “second hydrogen carrier production load range”) for the load (hereinafter also referred to as “second hydrogen carrier production load”) of the ammonia manufacturing device 22. The second hydrogen carrier production load range is defined by the upper and lower limits of the first hydrogen carrier production load.

The control information in the present embodiment further includes a stable operation range (hereinafter also referred to as “hydrogen tank pressure range”) for the filling pressure (hereinafter also referred to as “hydrogen tank pressure”) of the hydrogen tank 12. The hydrogen tank pressure range is defined by the upper and lower limits of the hydrogen tank pressure.

The information acquisition unit 101 acquires consumption area information about the consumption area of each type of hydrogen carrier. The consumption area information in the present embodiment includes the demand amount of hydrogen and each type of hydrogen carrier, the sales price of hydrogen and each type of hydrogen carrier, and the constraint conditions for delivering each type of hydrogen carrier.

The demand amount of hydrogen and each type of hydrogen carrier is the amount of hydrogen and each type of hydrogen carrier that is expected to be in demand at the consumption area R2 in a predetermined plan period. The demand amount of hydrogen and each type of hydrogen carrier can be acquired, for example, by inquiring of the past sales amount and the future purchase amount from the consumer C existing at the consumption area R2.

The sales price of hydrogen and each type of hydrogen carrier is the price of hydrogen and each type of hydrogen carrier that is expected to be available for sale at the consumption area R2 in a predetermined plan period. The sales price of hydrogen and each type of hydrogen carrier can be acquired, for example, by inquiring of the past sales price and the future purchase price from the consumer C existing at the consumption area R2.

The constraint conditions for delivering each type of hydrogen carrier include at least one of the constraint on the facility for receiving each type of hydrogen carrier at the consumption area R2 (facility constraint) or the constraint on transportation means for transporting each type of hydrogen carrier from the manufacturing area R1 to the consumption area R2 (transportation constraint). The facility constraint includes, for example, the capacity of the unloading pier, the capacity of a tank for storing the hydrogen carrier at the receiving area, the processing amount such as dehydrogenation in the hydrogen gas generation system 2, and the capacity of the liquid feeding or air feeding facilities. The transportation constraint includes, for example, the constraint on shipping such as the number of available tankers.

The sales planning unit 102 plans a sales plan for each type of hydrogen carrier during a predetermined plan period based on the consumption area information about each type of hydrogen carrier acquired by the information acquisition unit 101. The sales planning unit 102 stores information indicating the planned sales plan in a sales plan storage unit 121.

The sales planning unit 102 may generate information indicating the sales plan by presenting the consumption area information acquired by the information acquisition unit 101 to the user and receiving a sales plan input by the user. The sales planning unit 102 may generate information indicating the sales plan by applying predetermined rules to the consumption area information acquired by the information acquisition unit 101. These sales plan planning methods are examples. The sales planning unit 102 can generate information indicating a sales plan by any method for generating a sales plan based on the consumption area information.

For example, the sales planning unit 102 may increase the sales amount of each type of hydrogen carrier when the demand amount of hydrogen and each type of hydrogen carrier is expected to increase in the consumption area R2 or when the sales price of hydrogen and each type of hydrogen carrier is expected to increase in the consumption area R2. At this time, the sales planning unit 102 may determine the sales amount of each type of hydrogen carrier within the range that satisfies the supply capacity of each type of hydrogen carrier. When the sales amount is increased in the sales plan, the upper limit power price is set higher, and sales of hydrogen and each type of hydrogen carrier can be increased.

Also, for example, the sales planning unit 102 may decrease the sales amount of each type of hydrogen carrier when the demand amount of hydrogen and each type of hydrogen carrier is expected to decrease in the consumption area R2. At this time, the sales planning unit 102 may decrease the sales amount from the hydrogen carrier whose levelized cost of hydrogen calculated by the cost calculation unit 105 is high. When the sales amount is decreased in the sales plan, the upper limit power price is set lower, and the manufacturing cost of hydrogen and each type of hydrogen carrier can be reduced.

The power calculation unit 103 calculates the manufacturing amount of each type of hydrogen carrier based on the sales plan for each type of hydrogen carrier made by the sales planning unit 102. The power calculation unit 103 also calculates the total amount of power required (hereinafter also referred to as “total required power amount”) to satisfy the manufacturing amount of each type of hydrogen carrier.

The price calculation unit 104 calculates the average power price and the maximum power price when the total required power amount calculated by the power calculation unit 103 is satisfied based on the power market price profile stored in the predicted price storage unit 120.

The price calculation unit 104 calculates the average power price and the maximum power price while considering the rating ranges of the hydrogen manufacturing device 10, the MCH manufacturing device 21, and the ammonia manufacturing device 22. Further, when the power supplied from the power transmission grid G includes the power based on the power procurement contract with the fixed power price, the price calculation unit 104 calculates the average power price and the maximum power price while considering the fixed power price and the amount of power supplied under the power procurement contract. Further, the price calculation unit 104 calculates the average power price and the maximum power price so that variations in the manufacturing amount in the hydrogen manufacturing device 10 can be balanced out by the capacity of the hydrogen tank 12.

The cost calculation unit 105 calculates the levelized cost of hydrogen (LCoH) for each type of hydrogen carrier based on the average power price calculated by the price calculation unit 104. The levelized cost of hydrogen is the cost calculated from the total cost including the initial cost, operation cost, and disposal cost of the equipment for manufacturing each type of hydrogen carrier. The initial cost and disposal cost may be calculated in advance when the hydrogen carrier manufacturing system 1 is constructed. Among the operation costs, the costs due to transportation, dehydrogenation, etc., of each type of hydrogen carrier usually do not change significantly. However, these costs may be revised at appropriate timings such as when the equipment of the hydrogen gas generation system 2 is updated.

The threshold setting unit 106 calculates the difference between the levelized hydrogen cost calculated by the cost calculation unit 105 and the sales price of each type of hydrogen carrier planned by the sales planning unit 102 for each type of hydrogen carrier. The threshold setting unit 106 compares the difference between the levelized hydrogen cost and the sales price with a predetermined threshold. The predetermined threshold is calculated in advance in consideration of the target amount of profit to be obtained by the sales of the hydrogen carrier, the transportation cost for transporting each type of hydrogen carrier from the manufacturing area RI to the consumption area R2, and the processing cost for dehydrogenation and the like in the hydrogen gas generation system 2.

If the difference between the levelized hydrogen cost and the sales price for any type of hydrogen carrier is less than the threshold, the threshold setting unit 106 returns the processing to the sales planning unit 102 to reformulate the sales plan. On the other hand, if the difference between the levelized hydrogen cost and the sales price for all types of hydrogen carriers is greater than or equal to the threshold, the threshold setting unit 106 sets the maximum power price calculated by the price calculation unit 104 as the upper limit power price. The threshold setting unit 106 stores the set upper limit power price as control information in the control information storage unit 122.

The manufacturing amount setting unit 107 determines the upper limit manufacturing amount of each type of hydrogen carrier based on the manufacturing amount of each type of hydrogen carrier calculated by the power calculation unit 103. The manufacturing amount setting unit 107 stores the determined upper limit manufacturing amount of each type of hydrogen carrier as control information in the control information storage unit 122.

The price acquisition unit 108 acquires the power market price from the power market system M at predetermined time intervals. It is preferable that the time interval for acquiring the power market price matches the time interval for updating the power market price in the power market system M. In the present embodiment, the price acquisition unit 108 acquires the power market price every 5 minutes.

The load acquisition unit 109 acquires the hydrogen manufacturing load from the hydrogen manufacturing device 10. The hydrogen manufacturing load is, for example, a load factor of the hydrogen manufacturing device 10. The load factor of the hydrogen manufacturing device 10 is expressed as a ratio of the current output to the rated output of the hydrogen manufacturing device 10.

The load acquisition unit 109 acquires the first hydrogen carrier production load from the MCH manufacturing device 21. The first hydrogen carrier production load is, for example, a load factor of the MCH manufacturing device 21. The load factor of the MCH manufacturing device 21 is expressed as a ratio of the current output to the rated output of the MCH manufacturing device 21.

The load acquisition unit 109 acquires the second hydrogen carrier production load from the ammonia manufacturing device 22. The second hydrogen carrier production load is, for example, a load factor of the ammonia manufacturing device 22. The load factor of the ammonia manufacturing device 22 is expressed as a ratio of the current output to the rated output of the ammonia manufacturing device 22.

The pressure acquisition unit 110 acquires the hydrogen tank pressure from the hydrogen tank 12. The hydrogen tank pressure can be said to be information indicating the amount of hydrogen stored in the hydrogen tank 12.

The operation determination unit 111 determines the operation state of the hydrogen manufacturing device 10, the operation state of the MCH manufacturing device 21, and the operation state of the ammonia manufacturing device 22 based on the upper limit power price acquired by the price acquisition unit 108, the hydrogen manufacturing load, the first hydrogen carrier production load, and the second hydrogen carrier production load acquired by the load acquisition unit 109, and the control information stored in the control information storage unit 122.

The operation state of the hydrogen manufacturing device 10 includes the hydrogen manufacturing amount per unit time (hereinafter also referred to as “hydrogen manufacturing amount”). The operation state of the hydrogen manufacturing device 10 includes the operation mode of the hydrogen manufacturing device 10. The operation mode of the hydrogen manufacturing device 10 includes, for example, a hot standby mode. The hot standby mode is an operation state in which the hydrogen manufacturing device 10 itself is activated but hydrogen is not manufactured (that is, zero hydrogen manufacturing amount). When the hydrogen manufacturing device 10 is set to the hot standby mode, hydrogen manufacturing can be safely stopped while shortening the response time when hydrogen manufacturing is resumed.

The operation state of the MCH manufacturing device 21 includes the amount of methylcyclohexane manufactured per unit time (hereinafter also referred to as “first hydrogen conversion amount”). The operation state of the MCH manufacturing device 21 includes the operation mode of the MCH manufacturing device 21. The operation mode of the MCH manufacturing device 21 includes, for example, a hot recycling mode. The hot recycling mode is an operation state in which the MCH manufacturing device 21 itself is activated but methylcyclohexane is not manufactured (that is, the first hydrogen conversion amount is zero). When the MCH manufacturing device 21 is set to the hot recycling mode, it is possible to safely stop the manufacturing of methylcyclohexane while shortening the response time when the manufacturing of methylcyclohexane is resumed.

The operation state of the ammonia manufacturing device 22 includes the amount of ammonia manufactured per unit time (hereinafter also referred to as “second hydrogen conversion amount”). The operation state of the ammonia manufacturing device 22 includes the operation mode of the ammonia manufacturing device 22. The operation mode of the ammonia manufacturing device 22 includes, for example, a hot recycling mode. The hot recycling mode is an operation state in which the ammonia manufacturing device 22 itself is activated but ammonia is not manufactured (that is, the amount of second hydrogen conversion is zero). When the ammonia manufacturing device 22 is set to the hot recycling mode, it is possible to safely stop the manufacturing of ammonia while shortening the response time when the manufacturing of ammonia is resumed.

For example, when the hydrogen carrier is ammonia or liquid hydrogen, the safety and reliability may be affected if the ammonia manufacturing device 22 or the liquid hydrogen manufacturing device is automatically changed to the hot recycling mode. In the process of converting hydrogen to ammonia or liquid hydrogen, the processing conditions are severe such as high temperature, high pressure, or ultra-low temperature. Therefore, it is a matter to carefully decide whether to change the operation mode. Therefore, the designer can optionally decide whether to automatically change the ammonia manufacturing device 22 or the liquid hydrogen manufacturing device to the hot recycling mode based on the device design and the operation policy of the producer.

The device control unit 112 determines whether or not to change the hydrogen manufacturing amount, the first hydrogen conversion amount, or the second hydrogen conversion amount based on the operation state determined by the operation determination unit 111. When changing the hydrogen manufacturing amount, the device control unit 112 transmits a control signal for changing the power input to the hydrogen manufacturing device 10 to the power receiving and distributing facility 11.

When changing the first hydrogen conversion amount, the device control unit 112 transmits a control signal for changing the manufacturing amount of methylcyclohexane to the MCH manufacturing device 21. When the second hydrogen conversion amount is changed, the device control unit 112 transmits a control signal for changing the manufacturing amount of ammonia to the ammonia manufacturing device 22.

### Processing Procedure of the Setting Method

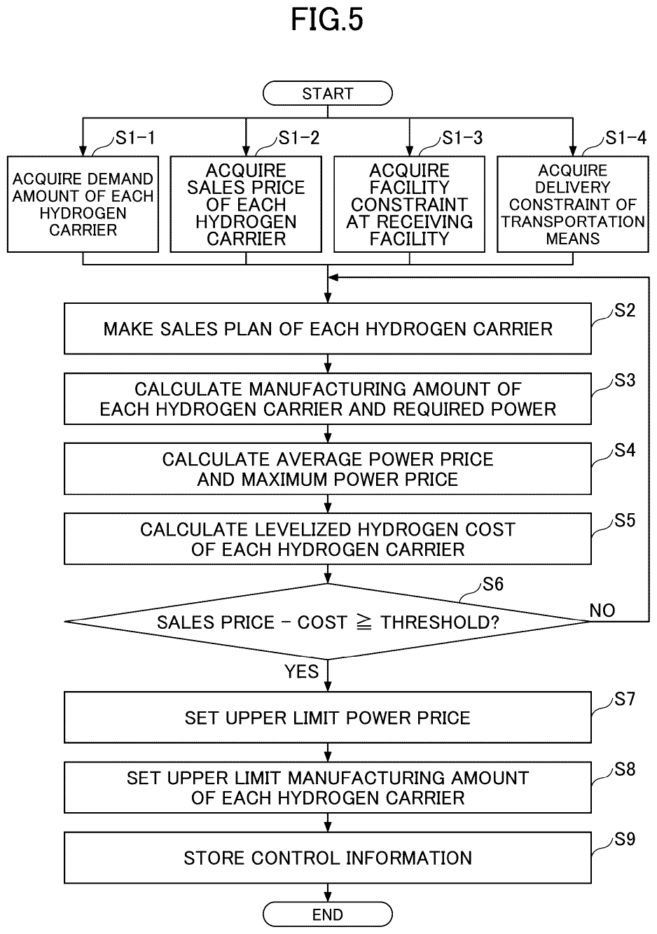

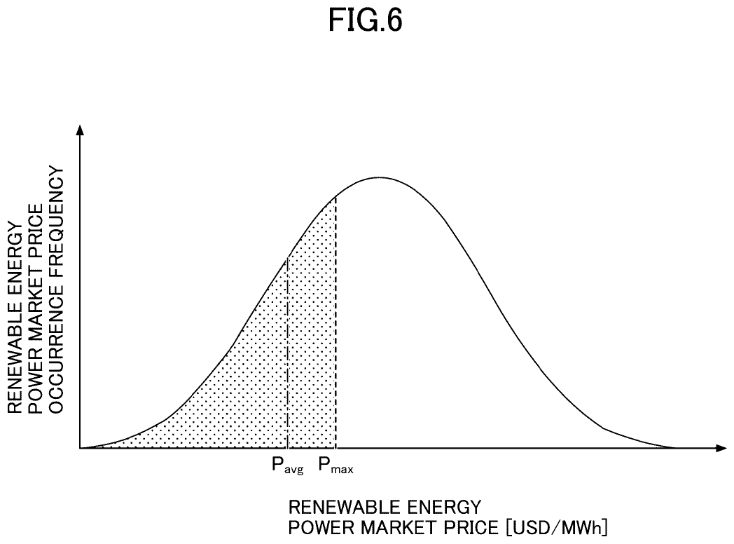

The setting method executed by the control apparatus 100 in the present embodiment will be described with reference to FIGS. 5 and 6. The setting method is a process for setting the control information based on the consumption area information. FIG. 5 is a flowchart illustrating an example of the setting method in the present embodiment.

In step S1-1, the information acquisition unit 101 of the control apparatus 100 acquires the demand amount of hydrogen and each type of hydrogen carrier in the consumption area R2. If there are a plurality of consumption areas R2, the information acquisition unit 101 acquires the demand amount of hydrogen and each type of hydrogen carrier for each of the plurality of consumption areas R2. The information acquisition unit 101 sends the information indicating the acquired demand amount of hydrogen and each type of hydrogen carrier to the sales planning unit 102.

In step S1-2, the information acquisition unit 101 of the control apparatus 100 acquires the sales price of hydrogen and each type of hydrogen carrier in the consumption area R2. If there are a plurality of consumption areas R2, the information acquisition unit 101 acquires the sales price of hydrogen and each type of hydrogen carrier for each of the plurality of consumption areas R2. The information acquisition unit 101 sends the information indicating the acquired sales price of hydrogen and each type of hydrogen carrier to the sales planning unit 102.

In step S1-3, the information acquisition unit 101 of the control apparatus 100 acquires the facility constraints related to hydrogen and each type of hydrogen carrier in the consumption area R2. If there are a plurality of consumption areas R2, the information acquisition unit 101 acquires the facility constraints for each of the plurality of consumption areas R2. The information acquisition unit 101 sends the information indicating the acquired facility constraint to the sales planning unit 102.

In step S1-4, the information acquisition unit 101 of the control apparatus 100 acquires the transportation constraint relating to hydrogen and each type of hydrogen carrier in the consumption area R2. If there are multiple consumption areas R2, the information acquisition unit 101 acquires the transportation constraint for each of the plurality of consumption areas R2. The information acquisition unit 101 sends the information indicating the acquired transportation constraint to the sales planning unit 102.

In step S2, the sales planning unit 102 of the control apparatus 100 receives the consumption area information relating to each type of hydrogen carrier from the information acquisition unit 101. The consumption area information includes information indicating the demand amount, sales price, facility constraint, and transportation constraint acquired in steps S1-1 to S1-4.

Next, the sales planning unit 102 makes a sales plan for each type of hydrogen carrier during a predetermined plan period based on the received consumption area information. Subsequently, the sales planning unit 102 stores information indicating the planned sales plan in the sales plan storage unit 121.

For example, the sales planning unit 102 may increase the sales amount of hydrogen carriers when the demand amount of hydrogen and each type of hydrogen carrier is expected to increase in the consumption area R2 or when the sales price of hydrogen and each type of hydrogen carrier is expected to increase in the consumption area R2. At this time, the sales planning unit 102 may determine the sales amount of hydrogen carriers within the range that satisfies the supply capacity (including manufacturing, transportation, dehydrogenation, etc.) of hydrogen carriers. If the sales amount increases in the sales plan, the upper limit power price is set higher, and the sales of hydrogen and each type of hydrogen carrier can be increased.

Also, for example, the sales planning unit 102 may decrease the sales amount of hydrogen carriers when the demand amount of hydrogen and each type of hydrogen carrier is expected to decrease in the consumption area R2. If the sales amount decreases in the sales plan, the upper limit power price is set lower, and the manufacturing cost of hydrogen and each type of hydrogen carrier can be reduced.

In step S3, the power calculation unit 103 of the control apparatus 100 reads the sales plan of each type of hydrogen carrier stored in the sales plan storage unit 121. Next, the power calculation unit 103 calculates the manufacturing amount of each type of hydrogen carrier based on the read sales plan. Next, the power calculation unit 103 calculates the total required power amount based on the calculated manufacturing amount of each type of hydrogen carrier.

The power calculation unit 103 calculates the manufacturing amount of each type of hydrogen carrier and the total required power amount while considering the efficiency of the hydrogen manufacturing device 10, the MCH manufacturing device 21, and the ammonia manufacturing device 22. The power calculation unit 103 calculates the required power amount by including the load of peripheral devices in addition to the hydrogen manufacturing device 10, the MCH manufacturing device 21, and the ammonia manufacturing device 22. The power calculation unit 103 then sends the calculated total required power amount to the price calculation unit 104. The power calculation unit 103 also sends the calculated manufacturing amount of each type of hydrogen carrier to the manufacturing amount setting unit 107.

In step S4, the price calculation unit 104 of the control apparatus 100 receives the manufacturing amount of each type of hydrogen carrier and the total required power amount from the power calculation unit 103. Next, the price calculation unit 104 reads the power market price profile stored in the predicted price storage unit 120.

Next, the price calculation unit 104 calculates the average power price and the maximum power price when the total required power amount is satisfied based on the read power market price profile. Next, the price calculation unit 104 sends the average power price to the cost calculation unit 105. The price calculation unit 104 also sends the maximum power price to the threshold setting unit 106.

### Calculation of Average Power Price and Upper Limit Power Price

The calculation method of average power price and upper limit power price in the present embodiment will be described with reference to FIG. 6. FIG. 6 is a diagram illustrating an example of the power market price profile in the present embodiment.

As illustrated in FIG. 6, the power market price profile in the present embodiment is a graph illustrating the frequency distribution of the power market price of renewable energy generated per unit time, with the horizontal axis representing the power market price of renewable energy [USD/MWh] and the vertical axis representing the power market price occurrence frequency of renewable energy. It is preferable that the unit time matches the plan period.

Because the amount of power derived from renewable energy fluctuates depending on the season and weather, the market price of power derived from renewable energy also fluctuates according to the amount of power generated. Therefore, the power market price of a specific month can be roughly predicted by performing statistical processing such as averaging on data obtained by recording the power market price for the past several years. The power market price may also be predicted by simulation or machine learning by using time-series data of power market price in the past.

In the calculation of the average power price and the maximum power price, the power market price occurrence frequency is calculated from the lower power price (left side of the graph) in the power market price profile. Because the power market price for one time means that power is purchased only at the interval of updating the power market price (for example, 5 minutes), the price when the power price occurrence frequency is integrated and the required power amount is reached, becomes the maximum power price Pmax for satisfying the required power amount. If the maximum power price Pmax is obtained, the average power price Pavy can be obtained from the shape of the power market price profile.

According to the above calculation method, the higher the sales amount of hydrogen carriers in the sales plan, the higher the maximum power price Pmax. As a result, sales of hydrogen and each type of hydrogen carrier can be increased. The sales amount of hydrogen carriers is set higher if demand for hydrogen and each type of hydrogen carrier is expected to increase in the consumption area R2 or if the sales price of hydrogen and each type of hydrogen carrier is expected to increase in the consumption area R2.

On the other hand, the lower the sales amount of hydrogen carriers in the sales plan, the lower the maximum power price Pmax. As a result, the manufacturing cost of hydrogen and each type of hydrogen carrier can be reduced. The sales amount of each type of hydrogen carrier is set to be small when the demand amount of hydrogen and each type of hydrogen carrier is expected to decrease in the consumption area R2.

Referring back to FIG. 5, the explanation will be given. In step S5, the cost calculation unit 105 of the control apparatus 100 receives the average power price from the price calculation unit 104. Next, the cost calculation unit 105 calculates the levelized hydrogen cost of each type of hydrogen carrier based on the average power price. Subsequently, the cost calculation unit 105 sends the levelized hydrogen cost of each type of hydrogen carrier to the threshold setting unit 106.

In step S6, the threshold setting unit 106 of the control apparatus 100 receives the levelized hydrogen cost of each type of hydrogen carrier from the cost calculation unit 105. Next, the threshold setting unit 106 reads the sales plan stored in the sales plan storage unit 121. Next, the threshold setting unit 106 obtains the sales price of each type of hydrogen carrier from the read sales plan.

Next, the threshold setting unit 106 calculates the difference between the levelized hydrogen cost and the sales price for each type of hydrogen carrier. Next, the threshold setting unit 106 compares the difference between the levelized hydrogen cost and the sales price for each type of hydrogen carrier with a predetermined threshold. If the difference between the levelized hydrogen cost and the sales price for any type of hydrogen carrier is less than the threshold (NO), the threshold setting unit 106 returns the process to step S2. On the other hand, if the difference between the levelized hydrogen cost and the sales price for all types of hydrogen carriers is greater than or equal to the threshold (YES), the threshold setting unit 106 advances the process to step S7.

In step S7, the threshold setting unit 106 of the control apparatus 100 receives the maximum power price from the price calculation unit 104. Next, the threshold setting unit 106 sets the maximum power price as the upper limit power price.

In step S8, the manufacturing amount setting unit 107 of the control apparatus 100 receives the manufacturing amount of each type of hydrogen carrier from the power calculation unit 103. Next, the manufacturing amount setting unit 107 determines the upper limit manufacturing amount of each type of hydrogen carrier based on the manufacturing amount of each type of hydrogen carrier. At this time, the manufacturing amount setting unit 107 determines the upper limit manufacturing amount of methylcyclohexane in consideration of the load fluctuation of the MCH manufacturing device 21. Similarly, the manufacturing amount setting unit 107 determines the upper limit manufacturing amount of ammonia in consideration of the load fluctuation of the ammonia manufacturing device 22.

In step S9, the threshold setting unit 106 of the control apparatus 100 stores the upper limit power price as control information in the control information storage unit 122. The manufacturing amount setting unit 107 of the control apparatus 100 stores the upper limit manufacturing amount of each type of hydrogen carrier as control information in the control information storage Each set value included in the control information may be reset at predetermined time intervals (for example, every few days). To determine whether or not to reset the value, the manufacturing amount of each type of hydrogen carrier calculated based on the sales plan may be compared with the manufacturing amount of each type of hydrogen carrier actually manufactured. For example, if the actual manufacturing amount greatly exceeds the planned manufacturing amount, the upper limit power price may be lowered. For example, if the balance of the manufacturing amount of each type of hydrogen carrier deviates from the demand amount, the upper limit manufacturing amount of each type of hydrogen carrier may be adjusted.

### Processing Procedure of the Control Method

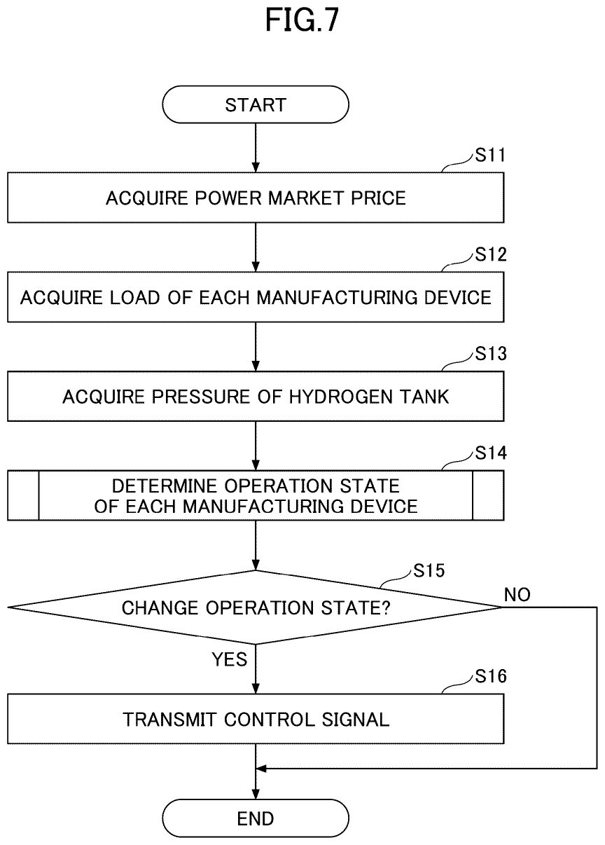

The control method executed by the control apparatus 100 in the present embodiment will be described with reference to FIGS. 7 to 12. The control method is a process for controlling each device for manufacturing hydrogen carriers based on the control information. FIG. 7 is a flowchart illustrating an example of the control method in the present embodiment.

In step S11, the price acquisition unit 108 of the control apparatus 100 acquires the power market price from the power market system M at predetermined time intervals. Next, the price acquisition unit 108 sends the power market price to the operation determination unit 111.

In step S12, the load acquisition unit 109 of the control apparatus 100 acquires the hydrogen manufacturing load from the hydrogen manufacturing device 10. The load acquisition unit 109 acquires the first hydrogen carrier production load from the MCH manufacturing device 21. Further, the load acquisition unit 109 acquires the second hydrogen carrier production load from the ammonia manufacturing device 22. Next, the load acquisition unit 109 sends the hydrogen manufacturing load, the first hydrogen carrier production load, and the second hydrogen carrier production load to the operation determination unit 111.

In step S13, the pressure acquisition unit 110 of the control apparatus 100 acquires the hydrogen tank pressure from the hydrogen tank 12. Next, the pressure acquisition unit 110 sends the hydrogen tank pressure to the operation determination unit 111.

In step S14, the operation determination unit 111 of the control apparatus 100 receives the power market price from the price acquisition unit 108. The operation determination unit 111 also receives the hydrogen manufacturing load, the first hydrogen carrier production load, and the second hydrogen carrier production load from the load acquisition unit 109. Further, the operation determination unit 111 receives the hydrogen tank pressure from the pressure acquisition unit 110. Next, the operation determination unit 111 reads the upper limit power price, the hydrogen manufacturing load range, the first hydrogen carrier production load range, the second hydrogen carrier production load range, and the hydrogen tank pressure range from the control information stored in the control information storage unit 122.

The operation determination unit 111 determines the operation state of the hydrogen manufacturing device 10, the operation state of the MCH manufacturing device 21, and the operation state of the ammonia manufacturing device 22 based on the power market price, the hydrogen manufacturing load, the first hydrogen carrier production load, the second hydrogen carrier production load, the hydrogen tank pressure, and the control information (upper limit power price, hydrogen manufacturing load range, first hydrogen carrier production load range, second hydrogen carrier production load range, and hydrogen tank pressure range). Next, the operation determination unit 111 sends the operation state of the hydrogen manufacturing device 10, the operation state of the MCH manufacturing device 21, and the operation state of the ammonia manufacturing device 22 to the device control unit 112.

### Operation State Determination Process

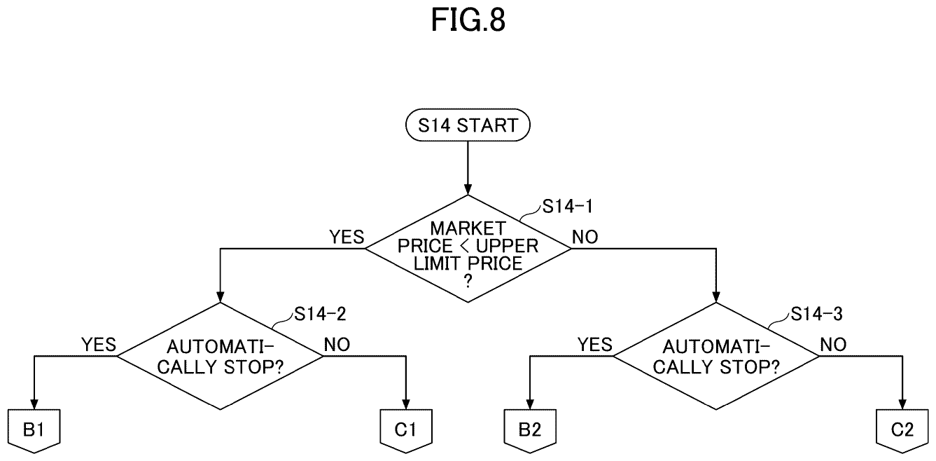

The operation state determination process (step S14 in FIG. 7) in the present embodiment will be described with reference to FIGS. 8 to 12. FIGS. 8 to 12 are flowcharts illustrating an example of the operation state determination process in the present embodiment. The operation state determination process is repeatedly executed for each type of hydrogen carrier manufacturing device whose operation state is to be determined.

In step S14-1, the operation determination unit 111 determines whether the power market price is lower than the upper limit power price. If the power market price is lower than the upper limit power price (YES), the operation determination unit 111 proceeds to step S14-2. On the other hand, if the power market price is greater than or equal to the upper limit power price (NO), the operation determination unit 111 proceeds to step S14-3.

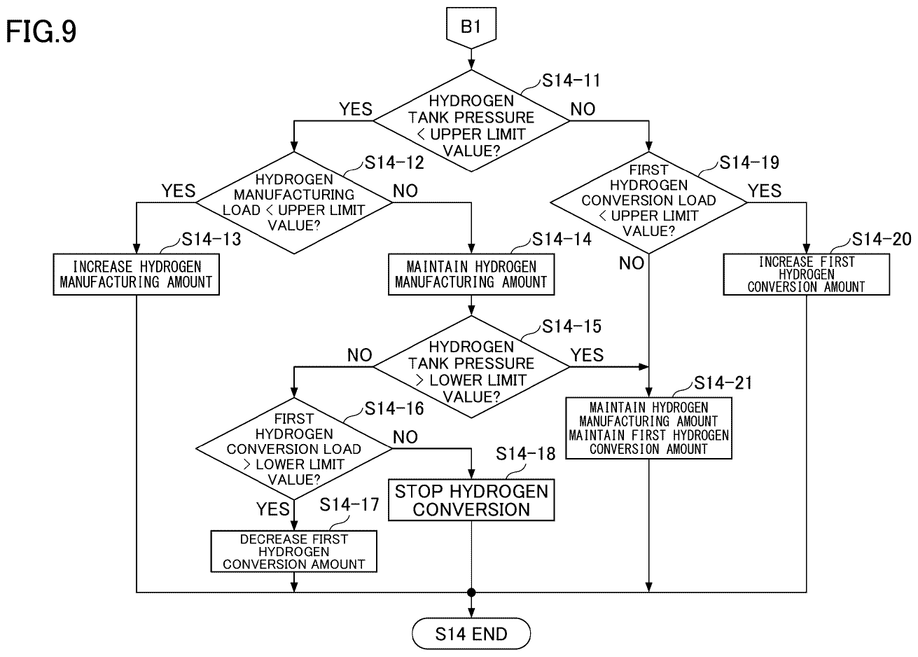

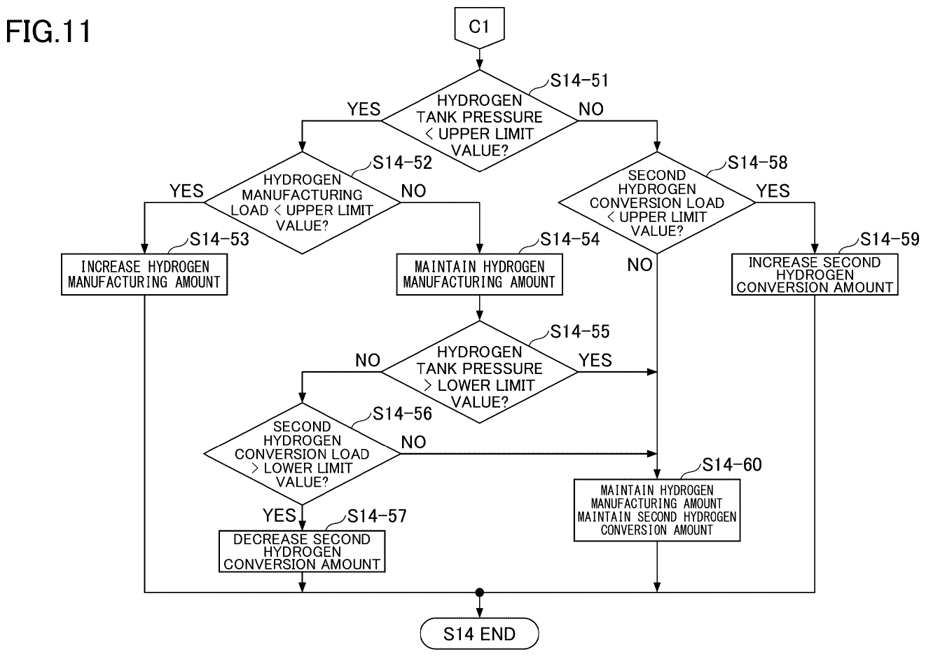

In step S14-2, the operation determination unit 111 determines whether or not to automatically shift the hydrogen carrier manufacturing device to the hot recycling mode. If the mode is to automatically shift to the hot recycling mode (YES), the operation determination unit 111 proceeds to step S14-11 (see FIG. 9). On the other hand, if the mode is not to automatically shift to the hot recycling mode (NO), the operation determination unit 111 proceeds to step S14-51 (see FIG. 11).

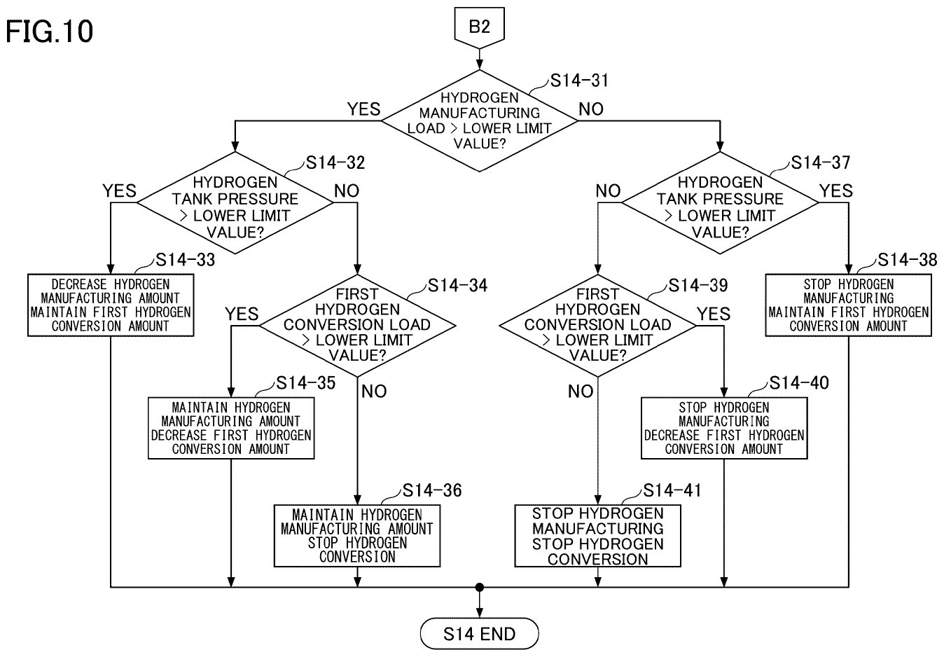

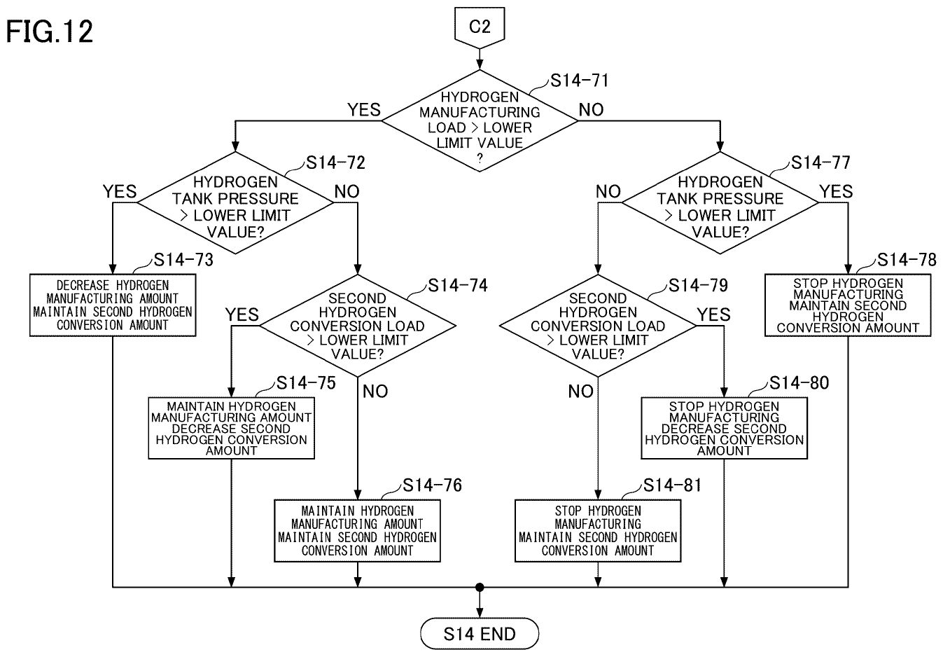

In step S14-3, the operation determination unit 111 determines whether or not to automatically shift the hydrogen carrier manufacturing device to the hot recycling mode. If the mode is to automatically shift to the hot recycling mode (YES), the operation determination unit 111 proceeds to step S14-31 (see FIG. 10). On the other hand, if the mode is not to automatically shift to the hot recycling mode (NO), the operation determination unit 111 proceeds to step S14-71 (see FIG. 12).

### If the Mode is to Automatically Shift to the Hot Recycling Mode

The operation state determination process when the hydrogen carrier manufacturing device is to be automatically shifted to the hot recycling mode will be described below with reference to FIGS. 9 and 10. Here, an example of automatically shifting the MCH manufacturing device 21 to the hot recycling mode will be described.

In step S14-11, the operation determination unit 111 determines whether the hydrogen tank pressure is lower than the upper limit of the hydrogen tank pressure range. If the hydrogen tank pressure is lower than the upper limit (YES), the operation determination unit 111 proceeds to step $14-12. On the other hand, if the hydrogen tank pressure is greater than or equal to the upper limit (NO), the operation determination unit 111 proceeds to step S14-19.

In step S14-12, the operation determination unit 111 determines whether the hydrogen manufacturing load is lower than the upper limit of the hydrogen manufacturing load range. If the hydrogen manufacturing load is lower than the upper limit (YES), the operation determination unit 111 proceeds to step S14-13. On the other hand, if the hydrogen manufacturing load is greater than or equal to the upper limit (NO), the operation determination unit 111 proceeds to step S14-14.

In step S14-13, the operation determination unit 111 increases the hydrogen manufacturing amount according to the difference between the power market price and the upper limit power price. Step S14-13 is executed when there is room to increase the hydrogen tank pressure and there is room to increase the hydrogen manufacturing load. In this case, the hydrogen manufacturing amount can be increased to increase sales.

In step S14-14, the operation determination unit 111 maintains the hydrogen manufacturing amount. Step S14-14 is executed when there is room to increase the hydrogen tank pressure but there is no room to increase the hydrogen manufacturing load. In this case, the hydrogen manufacturing amount can be maintained. Thereafter, the operation determination unit 111 proceeds to step S14-15.

In step S14-15, the operation determination unit 111 determines whether the hydrogen tank pressure is higher than the lower limit value (an example of the first lower limit amount) of the hydrogen tank pressure range. If the hydrogen tank pressure is higher than the lower limit value (YES), the operation determination unit 111 proceeds to step S14-21. On the other hand, if the hydrogen tank pressure is less than or equal to the lower limit value (NO), the operation determination unit 111 proceeds to step S14-16.

In step S14-16, the operation determination unit 111 determines whether the first hydrogen carrier production load is higher than the lower limit value (an example of the second lower limit amount) of the first hydrogen carrier production load range. If the first hydrogen carrier production load is higher than the lower limit value (YES), the operation determination unit 111 proceeds to step S14-17. On the other hand, if the first hydrogen carrier production load is less than or equal to the lower limit value (NO), the operation determination unit 111 proceeds to step S14-18.

In step S14-17, the operation determination unit 111 decreases the first hydrogen conversion amount according to the difference between the hydrogen tank pressure and the lower limit value of the hydrogen tank pressure range. Step S14-17 is executed when there is no room to decrease the hydrogen tank pressure and there is room to decrease the first hydrogen carrier production load. In this case, the first hydrogen conversion amount is decreased so that the hydrogen tank pressure is maintained at the lower limit.

In step S14-18, the operation determination unit 111 shifts the operation mode of the MCH manufacturing device 21 to the hot recycling mode. Step S14-18 is executed when there is no room to reduce the hydrogen tank pressure or the first hydrogen carrier production load. In this case, the manufacturing of methylcyclohexane is stopped so that the hydrogen tank pressure is restored to the lower limit or more.

In step S14-19, the operation determination unit 111 determines whether the first hydrogen carrier production load is lower than the upper limit of the first hydrogen carrier production load range. If the first hydrogen carrier production load is lower than the upper limit (YES), the operation determination unit 111 proceeds to step S14-20. On the other hand, if the first hydrogen carrier production load is greater than or equal to the upper limit (NO), the operation determination unit 111 proceeds to step S14-21.

In step S14-20, the operation determination unit 111 increases the first hydrogen conversion amount at a predetermined rate. Step S14-20 is executed when there is no room to increase the hydrogen tank pressure and there is room to increase the first hydrogen carrier production load. In this case, the first hydrogen conversion amount is increased so that the hydrogen tank pressure decreases to the upper limit value or less.

In step S14-21, the operation determination unit 111 maintains the hydrogen manufacturing amount and the first hydrogen conversion amount. Step S14-21 is executed in the following two cases when it is desired to increase the hydrogen carrier manufacturing amount because the power market price is lower than the upper limit power price. In the first case, there is room to change the hydrogen tank pressure, but there is no room to increase the hydrogen manufacturing load (YES in step S14-15). In the second case, there is no room to increase the hydrogen tank pressure and there is no room to increase the first hydrogen carrier production load (NO in step S14-19). In these cases, because there is no room to change the hydrogen manufacturing amount or the first hydrogen conversion amount, it is sufficient to maintain the hydrogen manufacturing amount and the first hydrogen conversion amount.

The explanation will proceed with FIG. 10. In step S14-31, the operation determination unit 111 determines whether the hydrogen manufacturing load is higher than the lower limit of the hydrogen manufacturing load range. If the hydrogen manufacturing load is higher than the lower limit (YES), the operation determination unit 111 proceeds to step $14-32. On the other hand, if the hydrogen manufacturing load is less than or equal to the lower limit (NO), the operation determination unit 111 proceeds to step S14-37.

In step S14-32, the operation determination unit 111 determines whether the hydrogen tank pressure is higher than the lower limit of the hydrogen tank pressure range (an example of the first lower limit). If the hydrogen tank pressure is higher than the lower limit (YES), the operation determination unit 111 proceeds to step S14-33. On the other hand, if the hydrogen tank pressure is less than or equal to the lower limit (NO), the operation determination unit 111 proceeds to step S14-34.

In step S14-33, the operation determination unit 111 reduces the hydrogen manufacturing amount according to the difference between the power market price and the upper limit power price. The operation determination unit 111 maintains the first hydrogen conversion amount. Step S14-33 is executed when there is room to reduce the hydrogen tank pressure and the hydrogen manufacturing load. In this case, the hydrogen manufacturing amount can be reduced and the manufacturing cost can be reduced.

In step S14-34, the operation determination unit 111 determines whether the first hydrogen carrier production load is higher than the lower limit of the first hydrogen carrier production load range (an example of the second lower limit). If the first hydrogen carrier production load is higher than the lower limit (YES), the operation determination unit 111 proceeds to step S14-35. On the other hand, if the first hydrogen carrier production load is less than or equal to the lower limit (NO), the operation determination unit 111 proceeds to step S14-36.

In step S14-35, the operation determination unit 111 maintains the hydrogen manufacturing amount. Further, the operation determination unit 111 decreases the first hydrogen conversion amount in accordance with the difference between the hydrogen tank pressure and the lower limit of the hydrogen tank pressure range. Step S14-35 is executed when there is no room to decrease the hydrogen tank pressure and there is room to decrease the first hydrogen carrier production load. In this case, the first hydrogen conversion amount is decreased while maintaining the hydrogen manufacturing amount so that the hydrogen tank pressure is recovered to the lower limit value or more.

In step S14-36, the operation determination unit 111 maintains the hydrogen manufacturing amount. Further, the operation determination unit 111 shifts the operation mode of the MCH manufacturing device 21 to the hot recycling mode. That is, the manufacturing of methylcyclohexane is stopped. Step S14-36 is executed when there is no room to decrease the hydrogen tank pressure and there is no room to decrease the first hydrogen carrier production load. In this case, the manufacturing of methylcyclohexane is stopped so that the hydrogen tank pressure is restored to the lower limit value or more.

In step S14-37, the operation determination unit 111 determines whether the hydrogen tank pressure is higher than the lower limit value of the hydrogen tank pressure range (an example of the first lower limit amount). If the hydrogen tank pressure is higher than the lower limit value (YES), the operation determination unit 111 proceeds to step S14-38. On the other hand, if the hydrogen tank pressure is less than or equal to the lower limit value (NO), the operation determination unit 111 proceeds to step S14-39.

In step S14-38, the operation determination unit 111 shifts the operation mode of the hydrogen manufacturing device 10 to the hot standby mode. That is, the manufacturing of hydrogen is stopped. The operation determination unit 111 maintains the first hydrogen conversion amount. Step S14-38 is executed when there is room to decrease the hydrogen tank pressure and there is no room to decrease the hydrogen manufacturing load. In this case, the manufacturing of methylcyclohexane can be continued while the manufacturing of hydrogen is stopped.

In step S14-39, the operation determination unit 111 determines whether the first hydrogen carrier production load is higher than the lower limit of the first hydrogen carrier production load range (an example of the second lower limit). If the first hydrogen carrier production load is higher than the lower limit (YES), the operation determination unit 111 advances the process to step S14-40. On the other hand, if the first hydrogen carrier production load is less than or equal to the lower limit (NO), the operation determination unit 111 advances the process to step S14-41.

In step S14-40, the operation determination unit 111 shifts the operation mode of the hydrogen manufacturing device 10 to the hot standby mode. That is, the manufacturing of hydrogen is stopped. Further, the operation determination unit 111 decreases the first hydrogen conversion amount according to the difference between the hydrogen tank pressure and the lower limit of the hydrogen tank pressure range. Step S14-40 is executed when there is no room to reduce the hydrogen manufacturing load but there is room to reduce the first hydrogen carrier production load. In this case, the manufacturing of methylcyclohexane can be continued while the manufacturing of hydrogen is stopped.

In step S14-41, the operation determination unit 111 shifts the operation mode of the hydrogen manufacturing device 10 to the hot standby mode. That is, the manufacturing of hydrogen is stopped. The operation determination unit 111 shifts the operation mode of the MCH manufacturing device 21 to the hot recycling mode. That is, the manufacturing of methylcyclohexane is stopped. Step S14-41 is executed when there is no room to reduce the hydrogen manufacturing load and there is no room to reduce the first hydrogen carrier production load. In this case, the manufacturing of hydrogen and the manufacturing of methylcyclohexane are stopped until the power market price falls to less than or equal to the upper limit power price.

### When the Hydrogen Carrier Manufacturing Device is Not Automatically Shifted to the Hot Recycling Mode

The operation state determination processing when the hydrogen carrier manufacturing device is not automatically shifted to the hot recycling mode will be described below with reference to FIGS. 11 and 12. Here, an example in which the ammonia manufacturing device 22 is not automatically shifted to the hot recycling mode will be described. Further, differences from the operation state determination processing when the hydrogen carrier manufacturing device is automatically shifted to the hot recycling mode (see FIGS. 9 and 10) will mainly be described.

The explanation will proceed with FIG. 11. In step S14-56, the operation determination unit 111 determines whether or not the second hydrogen carrier production load is higher than the lower limit of the second hydrogen carrier production load range (an example of the second lower limit). If the second hydrogen carrier production load is higher than the lower limit (YES), the operation determination unit 111 proceeds to step S14-57. On the other hand, if the second hydrogen carrier production load is less than or equal to the lower limit (NO), the operation determination unit 111 proceeds to step S14-60.

In step S14-60, the operation determination unit 111 maintains the hydrogen manufacturing amount and the second hydrogen conversion amount. Step S14-60 is executed in the following 3 cases in which it is desired to increase the manufacturing amount of the hydrogen carrier because the power market price is lower than the upper limit power price. In the first case, there is room to change the hydrogen tank pressure, but there is no room to increase the hydrogen manufacturing load (YES in step S14-55). In the second case, there is no room to increase the hydrogen tank pressure and there is no room to increase the second hydrogen carrier production load (NO in step S14-58). In the third case, there is no room to decrease the hydrogen tank pressure and no room to decrease the second hydrogen carrier production load (NO in step S14-56). Here, because the mode is not automatically shifted to the hot recycling mode, the manufacturing of hydrogen and the hydrogen carrier continues even in the third case.

The explanation will proceed with FIG. 12. In step S14-74, the operation determination unit 111 determines whether the second hydrogen carrier production load is higher than the lower limit value of the second hydrogen carrier production load range (an example of the second lower limit amount). If the second hydrogen carrier production load is higher than the lower limit value (YES), the operation determination unit 111 proceeds to step S14-75. On the other hand, if the second hydrogen carrier production load is less than or equal to the lower limit value (NO), the operation determination unit 111 proceeds to step S14-76.

In step S14-76, the operation determination unit 111 maintains the hydrogen manufacturing amount. The operation determination unit 111 maintains the second hydrogen conversion amount. Step S14-76 is executed when there is no room to reduce the hydrogen tank pressure or the second hydrogen carrier production load. In this case, the mode is not automatically shifted to the hot recycling mode, and the manufacturing of hydrogen and the hydrogen carrier is continued.

In step S14-79, the operation determination unit 111 determines whether the second hydrogen carrier production load is higher than the lower limit value of the second hydrogen carrier production load range (an example of the second lower limit amount). If the second hydrogen carrier production load is higher than the lower limit value (YES), the operation determination unit 111 proceeds to step S14-80. On the other hand, if the second hydrogen carrier production load is less than or equal to the lower limit value (NO), the operation determination unit 111 proceeds to step S14-81.

In step S14-81, the operation determination unit 111 shifts the operation mode of the hydrogen manufacturing device 10 to the hot standby mode. That is, the manufacturing of hydrogen is stopped. The operation determination unit 111 maintains the second hydrogen conversion amount. Step S14-81 is executed when there is no room to reduce the hydrogen manufacturing load or the second hydrogen carrier production load. In this case, the mode is not automatically shifted to the hot recycling mode, and hydrogen manufacturing is stopped While hydrogen carrier manufacturing is continued.

Referring back to FIG. 7, the following description will be given. In step S15, the device control unit 112 of the control apparatus 100 receives from the operation determination unit 111 the operation state of the hydrogen manufacturing device 10, the operation state of the MCH manufacturing device 21, and the operation state of the ammonia manufacturing device 22. Next, the device control unit 112 determines whether to change the hydrogen manufacturing amount, the first hydrogen conversion amount, and the second hydrogen conversion amount.

Whether to change the hydrogen manufacturing amount is determined based on whether the current hydrogen conversion amount is different from the new hydrogen manufacturing amount indicated by the operation state of the hydrogen manufacturing device 10. At this time, it may be determined that the hydrogen manufacturing amount is different when the difference between the current hydrogen manufacturing amount and the new hydrogen manufacturing amount is greater than or equal to a predetermined first threshold.

Whether or not to change the first hydrogen conversion amount is determined based on whether or not the current first hydrogen conversion amount is different from the new first hydrogen conversion amount indicated by the operation state of the MCH manufacturing device 21. At this time, it may be determined that the first hydrogen conversion amount is different when the difference between the current first hydrogen conversion amount and the new first hydrogen conversion amount is greater than or equal to the predetermined second threshold.

Whether or not to change the second hydrogen conversion amount is determined based on whether or not the current second hydrogen conversion amount is different from the new second hydrogen conversion amount indicated by the operation state of the ammonia manufacturing device 22. At this time, it may be determined that the second hydrogen conversion amount is different when the difference between the current second hydrogen conversion amount and the new second hydrogen conversion amount is greater than or equal to the predetermined third threshold.

If it is determined that any of the hydrogen manufacturing amount, the first hydrogen conversion amount, and the second hydrogen conversion amount is to be changed (YES), the device control unit 112 proceeds to step S16. On the other hand, if it is determined that none of the hydrogen manufacturing amount, the first hydrogen conversion amount, and the second hydrogen conversion amount is to be changed (NO), the device control unit 112 skips step S16 and ends the control method processing.

In step S16, when the hydrogen manufacturing amount is to be changed, the device control unit 112 of the control apparatus 100 transmits a control signal for changing the power input to the hydrogen manufacturing device 10, to the power receiving and distributing facility 11. The control signal includes information indicating the power after the change.

When changing the first hydrogen conversion amount, the device control unit 112 transmits a control signal for changing the manufacturing amount of methylcyclohexane to the MCH manufacturing device 21. The control signal includes information indicating the manufacturing amount after the change.

When changing the second hydrogen conversion amount, the device control unit 112 transmits a control signal for changing the manufacturing amount of ammonia to the ammonia manufacturing device 22. The control signal includes information indicating the manufacturing amount after the change.

The power receiving and distributing facility 11 receives a control signal from the control apparatus 100. Next, the power receiving and distributing facility 11 sets the power input to the hydrogen manufacturing device 10 based on the received control signal. The hydrogen manufacturing device 10 manufactures hydrogen according to the power input from the power receiving and distributing facility 11.

The MCH manufacturing device 21 receives a control signal from the control apparatus 100. Next, the MCH manufacturing device 21 sets the manufacturing amount of methylcyclohexane based on the received control signal, and manufactures methylcyclohexane so as to satisfy the manufacturing amount.

The ammonia manufacturing device 22 receives a control signal from the control apparatus 100. Next, the ammonia manufacturing device 22 sets the manufacturing amount of ammonia based on the received control signal, and manufactures ammonia so as to satisfy the manufacturing amount.

### Effect of Embodiment

The hydrogen carrier manufacturing system 1 includes the hydrogen manufacturing device 10 for manufacturing hydrogen by using power, the hydrogen tank 12 for storing hydrogen manufactured by the hydrogen manufacturing device 10, the MCH manufacturing device 21 for converting hydrogen stored in the hydrogen tank 12 into methylcyclohexane, and the ammonia manufacturing device 22 for converting hydrogen stored in the hydrogen tank 12 into ammonia. Therefore, according to the hydrogen carrier manufacturing system 1 of the present embodiment, a plurality of types of hydrogen carriers can be manufactured in parallel.

The hydrogen carrier manufacturing system 1 of the present embodiment further includes the control apparatus 100 for controlling the operation state of at least one of the hydrogen manufacturing device 10, the MCH manufacturing device 21, and the ammonia manufacturing device 22. Therefore, according to the hydrogen carrier manufacturing system 1 of the present embodiment, devices for manufacturing a plurality of types of hydrogen carriers can be controlled.

The control apparatus 100 of the present embodiment transmits a control signal for controlling the operation state of at least one of the hydrogen manufacturing device 10, the MCH manufacturing device 21, or the ammonia manufacturing device 22 based on the consumption area information about the consumption area R2. Therefore, according to the hydrogen carrier manufacturing system 1 of the present embodiment, devices for manufacturing a plurality of types of hydrogen carriers can be appropriately controlled based on the consumption area information.

Based on the consumption area information, the control apparatus 100 of the present embodiment sets a upper limit power price for determining whether or not to purchase the power for manufacturing at least one of hydrogen or hydrogen carriers at the manufacturing area R1, and transmits a control signal for controlling the operation state of at least one of the hydrogen manufacturing device 10, the MCH manufacturing device 21, or the ammonia manufacturing device 22 based on the comparison result of comparison between the power market price at the manufacturing area R1 and the upper limit power price. Therefore, according to the hydrogen carrier manufacturing system 1 of the present embodiment, a device for manufacturing at least one of hydrogen or a plurality of types of hydrogen carriers can be appropriately controlled based on the consumption area information and the power market price.

The control apparatus 100 in the present embodiment determines the manufacturing amount of each type of hydrogen carrier based on the consumption area information. The control apparatus 100 determines the manufacturing amount of each type of hydrogen carrier based on the respective loads of the hydrogen manufacturing device 10, the MCH manufacturing device 21, and the ammonia manufacturing device 22, and the storage amount of the hydrogen tank 12. Therefore, according to the hydrogen carrier manufacturing system 1 in the present embodiment, the manufacturing amount of a plurality of types of hydrogen carriers can be appropriately determined based on the load of the device for manufacturing hydrogen carriers.

The control apparatus 100 in the present embodiment performs control to increase the manufacturing amount of hydrogen when the power market price is lower than the upper limit power price and the filling pressure of the hydrogen tank 12 is lower than the predetermined upper limit value. The control apparatus 100 performs control to increase the manufacturing amount of hydrogen carriers when the power market price is lower than the upper limit power price and the filling pressure of the hydrogen tank 12 is higher than the predetermined upper limit value. Therefore, according to the control apparatus 100 in the present embodiment, the manufacturing amount of hydrogen and hydrogen carriers can be increased as long as the filling pressure of the hydrogen tank 12 is within the stable operation range.

The control apparatus 100 in the present embodiment performs control to decrease the manufacturing amount of hydrogen and maintain the manufacturing amount of hydrogen carriers when the power market price is higher than the upper limit power price and the filling pressure of the hydrogen tank 12 is higher than the predetermined lower limit value. When the power market price is higher than the upper limit power price and the filling pressure of the hydrogen tank 12 is lower than the predetermined lower limit value, the control apparatus 100 performs control to maintain the manufacturing amount of hydrogen and to decrease the manufacturing amount of hydrogen carrier. Therefore, according to the control apparatus 100 of the present embodiment, the manufacturing amount of hydrogen and hydrogen carrier can be decreased as long as the filling pressure of the hydrogen tank 12 is within the stable operation range.

The control apparatus 100 of the present embodiment shifts the hydrogen manufacturing device 10 to the hot standby mode when the power market price is higher than the upper limit power price and the manufacturing amount of hydrogen is lower than the predetermined lower limit value. Therefore, according to the control apparatus 100 of the present embodiment, the manufacturing amount of hydrogen can be safely stopped so that the manufacturing amount of hydrogen does not fall below the lower limit value.

When the mode is to be automatically shifted to the hot recycling mode, the control apparatus 100 of the present embodiment causes the device for manufacturing hydrogen carriers to shift to the hot recycling mode when the power market price is higher than the upper limit power price, the filling pressure of the hydrogen tank 12 is less than or equal to the predetermined lower limit value, and the manufacturing amount of hydrogen carrier is less than or equal to the predetermined lower limit value. Therefore, according to the control apparatus 100 of the present embodiment, the manufacturing of hydrogen carriers can be safely stopped so that the filling pressure of the hydrogen tank 12 does not fall below the lower limit value.

When the mode is not to be automatically shifted to the hot recycling mode, the control apparatus 100 of the present embodiment maintains the manufacturing amount of hydrogen carriers regardless of the power market price, when the filling pressure of the hydrogen tank 12 is less than or equal to the predetermined lower limit value and the manufacturing amount of hydrogen carriers is less than or equal to the predetermined lower limit value. Therefore, according to the control apparatus 100 of the present embodiment, the manufacturing of hydrogen carriers can be continued so that the load of the hydrogen carrier manufacturing device 20 does not fall below the lower limit.

Therefore, according to the hydrogen carrier manufacturing system 1 of the present embodiment, the amount of purchased power can be precisely varied according to the power market price fluctuating every few minutes. Further, hydrogen carriers can be appropriately manufactured according to the market price of hydrogen, the amount of demand, the cost of the dehydrogenation treatment, etc., in the consumption area R2. For example, if the sales price increases or the demand increases in the consumption area R2, the manufacturing of hydrogen carriers can be increased considering the supply capacity (manufacturing, transportation, dehydrogenation, etc.) of the entire supply chain, Further, for example, if the demand decreases in the consumption area R2, the manufacturing of hydrogen carriers can be adjusted based on the levelized hydrogen cost.

In particular, according to the hydrogen carrier manufacturing system 1 of the present embodiment, the economic efficiency of each type of hydrogen carrier can be individually applied in the MCH manufacturing device 21 and the ammonia manufacturing device 22 (or the liquid hydrogen manufacturing device) having the hydrogen manufacturing device 10 as a common facility, and the total margin can be maximized. Further, each type of hydrogen carrier can be appropriately manufactured according to the market price of hydrogen, the amount of demand, the cost of dehydrogenation treatment, and the like in the consumption area R2. For example, if the demand decreases in the consumption area R2, the manufacturing of each type of hydrogen carrier can be adjusted in descending order according to the levelized hydrogen cost. Further, when the sales price and demand of each type of hydrogen carrier in the consumption area R2 are different, the amount of power purchased can be optimized by making a sales plan for each type of hydrogen carrier and adding them together.

### MODIFIED EXAMPLE 1

In the embodiment, the control apparatus 100 installed in the manufacturing area RI controls each device included in the hydrogen carrier manufacturing system 1 based on the consumption area information acquired from the consumption area R2. In modified example 1, the control apparatus installed in the consumption area R2 transmits the consumption area information to the control apparatus 100.

### Overall Configuration of the Hydrogen Gas Generation System

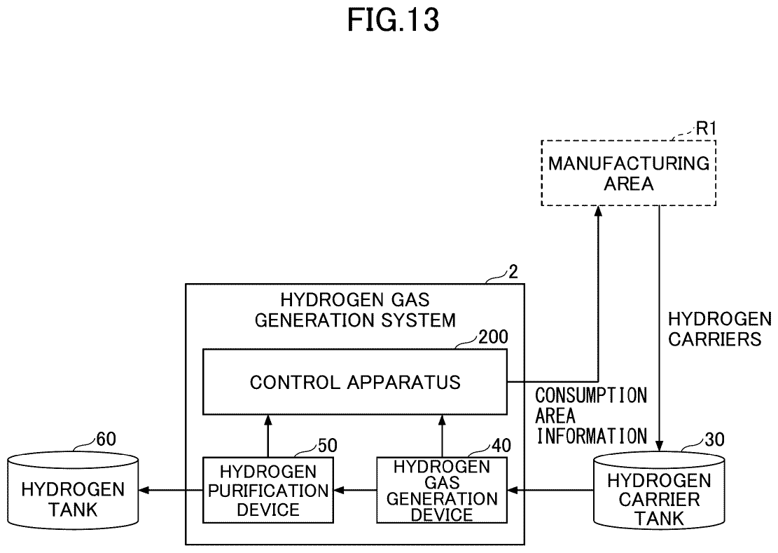

The overall configuration of the hydrogen gas generation system 2 in this modified example will be described with reference to FIG. 13. FIG. 13 is a block diagram illustrating an example of the overall configuration of the hydrogen gas generation system in the present embodiment.

As illustrated in FIG. 13, the hydrogen gas generation system 2 in this modified example includes a hydrogen gas generation device 40, a hydrogen purification device 50, and a control apparatus 200. The hydrogen gas generation system 2 in this modified example is connected to the hydrogen carrier tank 30 and a hydrogen tank 60 in the consumption area R2.

The control apparatus 200 is an information processing apparatus such as a personal computer, a workstation, or a server that controls the operation of each device included in the hydrogen gas generation system 2. The control apparatus 200 is configured to enable mutual data communication with the hydrogen gas generation device 40 and the hydrogen purification device 50 via a communication network. The control apparatus 200 acquires the consumption area information about the consumption area R2 and transmits it to the control apparatus 100 of the hydrogen carrier manufacturing system 1.

### Functional Configuration of the Hydrogen Gas Generation System

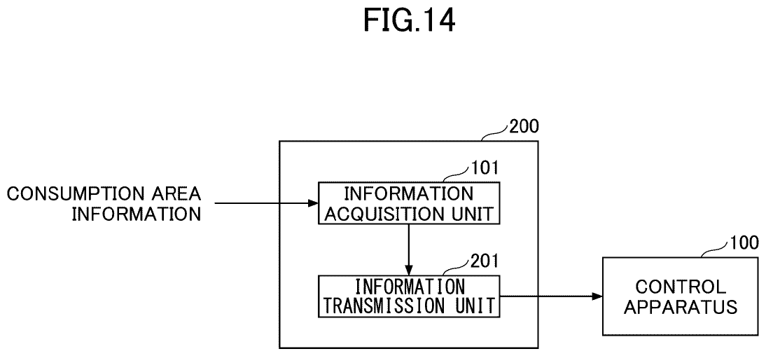

The functional configuration of the hydrogen gas generation system in this modified example will be described with reference to FIG. 14. FIG. 14 is a block diagram illustrating an example of the functional configuration of the hydrogen gas generation system 2 in this modified example.

### Functional Configuration of the Control Apparatus

As illustrated in FIG. 14, the control apparatus 200 in this modified example includes an information acquisition unit 101 and an information transmission unit 201. The information acquisition unit 101 functions similarly to the information acquisition unit 101 provided in the control apparatus 100 in the embodiment.

The information transmission unit 201 transmits the consumption area information obtained by the information acquisition unit 101 to the control apparatus 100 of the hydrogen carrier manufacturing system 1.

In the hydrogen carrier manufacturing system 1, the information acquisition unit 101 provided in the control apparatus 100 receives the consumption area information from the control apparatus 200 to acquire the consumption area information. The control apparatus 100 controls each device included in the hydrogen carrier manufacturing system 1 based on the consumption area information in the same manner as the control apparatus 100 in the embodiment.

### MODIFIED EXAMPLE 2

In modified example 1, the control apparatus 200 installed in the consumption area R2 transmits the consumption area information to the control apparatus 100. In modified example 2, the control apparatus installed in the consumption area R2 is configured to transmit control information to the control apparatus 100.

### Functional Configuration of the Hydrogen Gas Generation System

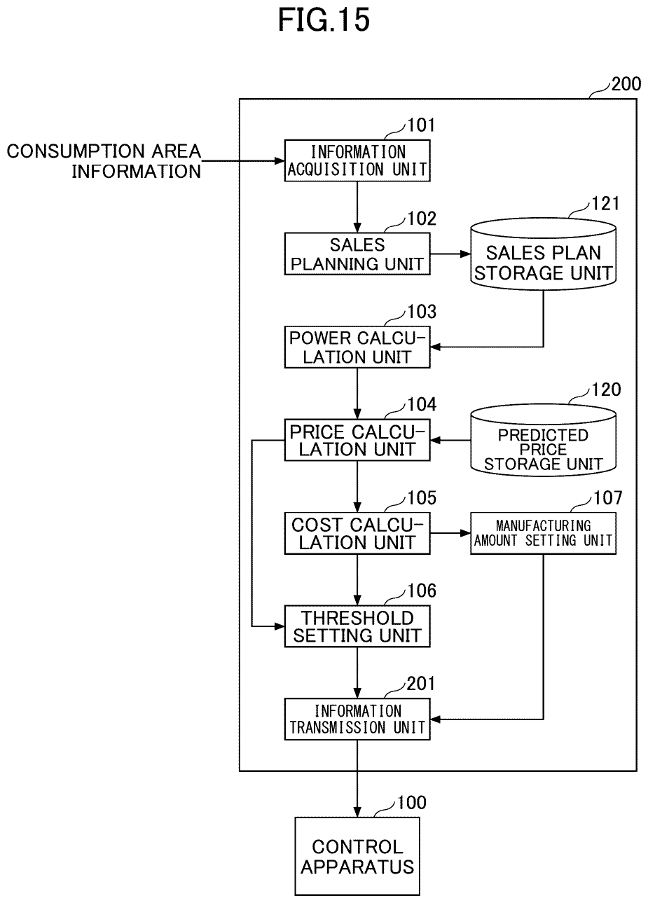

The functional configuration of the hydrogen gas generation system in this modified example will be described with reference to FIG. 15. FIG. 15 is a block diagram illustrating an example of the functional configuration of the hydrogen gas generation system 2 in this modified example.

### Functional Configuration of the Control Apparatus

As illustrated in FIG. 15, the control apparatus 200 in this modified example includes an information acquisition unit 101, a sales planning unit 102, a power calculation unit 103, a price calculation unit 104, a cost calculation unit 105, a threshold setting unit 106, a manufacturing amount setting unit 107, a predicted price storage unit 120, a sales plan storage unit 121, and an information transmission unit 201. That is, the control apparatus 200 in this modified example is different from the control apparatus 100 in the first embodiment in that it does not include the price acquisition unit 108, the load acquisition unit 109, the pressure acquisition unit 110, the operation determination unit 111, the device control unit 112, or the control information storage unit 122, but includes an information transmission unit 201.

The information transmission unit 201 in this modified example transmits control information including the upper limit power price set by the threshold setting unit 106 and the upper limit manufacturing amount of each type of hydrogen carrier set by the manufacturing amount setting unit 107, to the control apparatus 100 of the hydrogen carrier manufacturing system 1.

In the hydrogen carrier manufacturing system 1, the control apparatus 100 receives control information from the control apparatus 200 and stores it in the control information storage unit 122. The control apparatus 100 controls each device included in the hydrogen carrier manufacturing system 1 based on the control information read from the control information storage unit 122 as in the case of the control apparatus 100 in the embodiment.

### Application Example

The control apparatus 100 in the embodiment can be applied not only to the hydrogen carrier manufacturing system 1 in the operation stage but also to the hydrogen carrier manufacturing system 1 in the planning stage. That is, if the consumption area information about the consumption area and the estimated value of the power market price can be obtained, each set value included in the control information can be set, and the total margin in the case of operating based on the control information can be estimated. Therefore, the control apparatus 100 in the embodiment can be applied to maximize the economic efficiency of the hydrogen carrier manufacturing system 1 in the planning stage and optimize the investment scope.

### Note

Each of the functions of the above-described embodiments can be implemented by one or more processing circuits. Here, the term “processing circuit” as used herein includes a processor programmed to execute each function by software such as a CPU (Central Processing Unit) or a GPU (Graphics Processing Unit) implemented by an electronic circuit, and equipment such as an ASIC (Application Specific Integrated Circuit), DSP (Digital Signal Processor), FPGA (Field Programmable Gate Array), or a conventional circuit module designed to execute each of the above-described functions.

Note that, in the disclosed technology, modes as described in the clauses below can be considered.

### Clause 1

A hydrogen carrier manufacturing system including:
a hydrogen manufacturing device configured to manufacture hydrogen;
a hydrogen tank configured to store the hydrogen manufactured by the hydrogen manufacturing device; and
a plurality of hydrogen carrier manufacturing devices configured to convert the hydrogen stored in the hydrogen tank into different types of hydrogen carriers.

### Clause 2

The hydrogen carrier manufacturing system according to clause 1, wherein the plurality of hydrogen carrier manufacturing devices convert the hydrogen stored in the same hydrogen tank into the hydrogen carriers.

### Clause 3

The hydrogen carrier manufacturing system according to clause 1, further including:
a control apparatus configured to control an operation state of at least one of the hydrogen manufacturing device or the plurality of hydrogen carrier manufacturing devices.

### Clause 4

The hydrogen carrier manufacturing system according to clause 3, wherein the control apparatus includes:
an information acquisition unit configured to acquire consumption area information relating to a consumption area at which at least one of the hydrogen or the hydrogen carrier is consumed; and
a device control unit configured to control the operation state of at least one of the hydrogen manufacturing device or the plurality of hydrogen carrier manufacturing devices based on the consumption area information.

### Clause 5

The hydrogen carrier manufacturing system according to clause 4, wherein the control apparatus includes:
a price acquisition unit configured to acquire a power price at a manufacturing area at which the hydrogen carrier is manufactured; and
a threshold setting unit configured to set a threshold for determining whether or not to purchase power for manufacturing at least one of the hydrogen or the hydrogen carrier at the manufacturing area at which at least one of the hydrogen or the hydrogen carrier is manufactured, based on the consumption area information, wherein
the device control unit controls the operation state of at least one of the hydrogen manufacturing device or the plurality of hydrogen carrier manufacturing devices based on a comparison result of comparison between the power price and the threshold.

### Clause 6

The hydrogen carrier manufacturing system according to clause 4, wherein the consumption area information includes a demand amount of at least one of the hydrogen or the hydrogen carrier at the consumption area.

### Clause 7

The hydrogen carrier manufacturing system according to clause 4, wherein the consumption area information includes a sales price of at least one of the hydrogen or the hydrogen carrier at the consumption area.

### Clause 8

The hydrogen carrier manufacturing system according to clause 4, wherein the consumption area information includes a constraint condition for delivering the hydrogen carrier to the consumption area.

### Clause 9

The hydrogen carrier manufacturing system according to clause 8, wherein the constraint condition includes at least one of a constraint relating to a facility for receiving the hydrogen carrier at the consumption area or a constraint relating to a transportation means for transporting the hydrogen carrier from the manufacturing area where the hydrogen carrier is manufactured to the consumption area.

### Clause 10

The hydrogen carrier manufacturing system according to clause 4, wherein the control apparatus further includes:
an operation determination unit configured to determine a manufacturing amount of each of the plurality of hydrogen carrier manufacturing devices.

### Clause 11

The hydrogen carrier manufacturing system according to clause 10, wherein the operation determination unit determines the manufacturing amount of each of the plurality of hydrogen carrier manufacturing devices based on a load of the hydrogen manufacturing device, a load of each of the plurality of hydrogen carrier manufacturing devices, and a storage amount of the hydrogen tank.

### Clause 12

The hydrogen carrier manufacturing system according to clause 11, wherein when the storage amount of the hydrogen tank is less than or equal to a predetermined first lower limit value, the device control unit instructs to stop manufacturing of the hydrogen carrier by the hydrogen carrier manufacturing device whose manufacturing amount is less than or equal to a predetermined second lower limit value.

### Clause 13

The hydrogen carrier manufacturing system according to clause 12, wherein the device control unit instructs to stop manufacturing of methylcyclohexane when the manufacturing amount of the hydrogen carrier converting device for converting the hydrogen into the methylcyclohexane is less than or equal to the second lower limit value, and instructs to maintain a manufacturing amount of ammonia or liquid hydrogen when the manufacturing amount of the hydrogen carrier converting device for converting the hydrogen into the ammonia or the liquid hydrogen is less than or equal to the second lower limit value.

### Clause 14

A control apparatus configured to communicate with a hydrogen manufacturing device configured to manufacture hydrogen, a hydrogen tank configured to store the hydrogen manufactured by the hydrogen manufacturing device, and a plurality of hydrogen carrier manufacturing devices configured to convert the hydrogen stored in the hydrogen tank into different types of hydrogen carriers, wherein
the control apparatus controls an operation state of at least one of the hydrogen manufacturing device or the plurality of hydrogen carrier manufacturing devices.

### Clause 15

A hydrogen carrier manufacturing method including:
manufacturing hydrogen by a hydrogen manufacturing device;
storing the hydrogen manufactured by the hydrogen manufacturing device, in a hydrogen tank; and
converting the hydrogen stored in the hydrogen tank into different types of hydrogen carriers, by a plurality of hydrogen carrier manufacturing devices.

Although the embodiments of the present invention have been described in detail above, the present invention is not limited to these embodiments, and various modifications or changes may be made within the scope of the gist of the present invention as described in the claims.

This application is based upon and claims priority to Japanese Patent Application No. 2023-25202, filed on Feb. 21, 2023 before the Japan Patent Office, the entire contents of which are incorporated herein by reference.

### DESCRIPTION OF REFERENCE NUMERALS

1 hydrogen carrier manufacturing system
2 hydrogen gas generation system
10 hydrogen manufacturing device
11 power receiving and distributing facility
12 hydrogen tank
21 MCH manufacturing device
22 ammonia manufacturing device
31 MCH tank
32 ammonia tank
100 control apparatus
101 information acquisition unit
102 sales planning unit
103 power calculation unit
104 price calculation unit
105 cost calculation unit
106 threshold setting unit
107 manufacturing amount setting unit
108 price acquisition unit
109 load acquisition unit
110 pressure acquisition unit
111 operation determination unit
112 device control unit
120 predicted price storage unit
121 sales plan storage unit
122 control information storage unit
200 control apparatus
201 information transmission unit

1. A hydrogen carrier manufacturing system comprising:
a processor; and
a memory that includes instructions, which when executed, cause the processor to execute:
manufacturing hydrogen by a hydrogen manufacturing device;
storing, in a hydrogen tank, the hydrogen manufactured by the hydrogen manufacturing device; and
converting, by a plurality of hydrogen carrier manufacturing devices, the hydrogen stored in the hydrogen tank into different types of hydrogen carriers.

2. The hydrogen carrier manufacturing system according to claim 1, wherein the converting by the plurality of hydrogen carrier manufacturing devices includes converting the hydrogen stored in the hydrogen tank into the hydrogen carriers, the hydrogen tank being a same tank.

3. The hydrogen carrier manufacturing system according to claim 1, wherein the instructions, which when executed, cause the processor to execute:
controlling, by a control apparatus, an operation state of at least one of the hydrogen manufacturing device or the plurality of hydrogen carrier manufacturing devices.

4. The hydrogen carrier manufacturing system according to claim 3, wherein the controlling of the operation state by the control apparatus includes:
acquiring consumption area information relating to a consumption area at which at least one of the hydrogen or the hydrogen carrier is consumed; and
controlling the operation state of at least one of the hydrogen manufacturing device or the plurality of hydrogen carrier manufacturing devices based on the consumption area information.

5. The hydrogen carrier manufacturing system according to claim 4, wherein the controlling of the operation state by the control apparatus includes:
acquiring a power price at a manufacturing area at which the hydrogen carrier is manufactured; and
setting a threshold for determining whether or not to purchase power for manufacturing at least one of the hydrogen or the hydrogen carrier at the manufacturing area at which at least one of the hydrogen or the hydrogen carrier is manufactured, based on the consumption area information, wherein
the controlling includes controlling the operation state of at least one of the hydrogen manufacturing device or the plurality of hydrogen carrier manufacturing devices based on a comparison result of comparison between the power price and the threshold.

6. The hydrogen carrier manufacturing system according to claim 4, wherein the consumption area information includes a demand amount of at least one of the hydrogen or the hydrogen carrier at the consumption area.

7. The hydrogen carrier manufacturing system according to claim 4, wherein the consumption area information includes a sales price of at least one of the hydrogen or the hydrogen carrier at the consumption area.

8. The hydrogen carrier manufacturing system according to claim 4, wherein the consumption area information includes a constraint condition for delivering the hydrogen carrier to the consumption area.

9. The hydrogen carrier manufacturing system according to claim 8, wherein the constraint condition includes at least one of a constraint relating to a facility for receiving the hydrogen carrier at the consumption area or a constraint relating to a transportation means for transporting the hydrogen carrier from the manufacturing area where the hydrogen carrier is manufactured to the consumption area.

10. The hydrogen carrier manufacturing system according to claim 4, wherein the controlling of the operation state by the control apparatus further includes:
determining a manufacturing amount of each of the plurality of hydrogen carrier manufacturing devices.

11. The hydrogen carrier manufacturing system according to claim 10, wherein the determining includes determining the manufacturing amount of each of the plurality of hydrogen carrier manufacturing devices based on a load of the hydrogen manufacturing device, a load of each of the plurality of hydrogen carrier manufacturing devices, and a storage amount of the hydrogen tank.

12. The hydrogen carrier manufacturing system according to claim 11, wherein the controlling includes, when the storage amount of the hydrogen tank is less than or equal to a predetermined first lower limit value, instructing to stop manufacturing of the hydrogen carrier by the hydrogen carrier manufacturing device whose manufacturing amount is less than or equal to a predetermined second lower limit value.

13. The hydrogen carrier manufacturing system according to claim 12, wherein the controlling includes instructing to stop manufacturing of methylcyclohexane when the manufacturing amount of the hydrogen carrier converting device for converting the hydrogen into the methylcyclohexane is less than or equal to the second lower limit value, and instructing to maintain a manufacturing amount of ammonia or liquid hydrogen when the manufacturing amount of the hydrogen carrier converting device for converting the hydrogen into the ammonia or the liquid hydrogen is less than or equal to the second lower limit value.

14. A control apparatus configured to communicate with a hydrogen manufacturing device configured to manufacture hydrogen, a hydrogen tank configured to store the hydrogen manufactured by the hydrogen manufacturing device, and a plurality of hydrogen carrier manufacturing devices configured to convert the hydrogen stored in the hydrogen tank into different types of hydrogen carriers, the control apparatus comprising:
a processor; and
a memory that includes instructions, which when executed, cause the processor to execute:
controlling an operation state of at least one of the hydrogen manufacturing device or the plurality of hydrogen carrier manufacturing devices.

15. A hydrogen carrier manufacturing method comprising:
manufacturing hydrogen by a hydrogen manufacturing device;
storing the hydrogen manufactured by the hydrogen manufacturing device, in a hydrogen tank; and
converting the hydrogen stored in the hydrogen tank into different types of hydrogen carriers, by a plurality of hydrogen carrier manufacturing devices.
